(19) United States
(12) Patent Application Publication
(10) Pub. No.: US 2011/0045092 A1
(43) Pub. Date: Feb. 24, 2011

(54) CASEIN PARTICLES ENCAPSULATING THERAPEUTICALLY ACTIVE AGENTS AND USES THEREOF

(76) Inventors: Yoav D. Livney, Misgav (IL); Yehuda G. Assaraf, Misgav (IL); Alina Shapira, Naharia (IL)

Correspondence Address: Browdy and Neimark, PLLC, 1625 K Street, N.W., Suite 1100, Washington, DC, 20006, US

(21) Appl. No.: 12/867,219

(22) PCT Filed: Feb. 11, 2009
(86) PCT No.: PCT/IL09/00156

§ 371 Date: Nov. 1, 2010

Related U.S. Application Data
(60) Provisional application No. 61/027,645, filed on Feb. 11, 2008.

(51) Int. Cl. A61K 9/14; A61K 31/337; A61K 31/437; A61K 31/138; A61P 35/00; B82Y 5/00

(52) U.S. Cl. 424/491; 514/449; 514/283; 514/653; 977/773

(57) ABSTRACT

Casein particles, such as micelles or clusters thereof, having encapsulated therein hydrophobic and/or water insoluble therapeutically active agents such as hydrophobic chemotherapeutic agents, which are otherwise administered parenterally, are disclosed. Pharmaceutical compositions containing the casein particles and uses thereof in the treatment of cancer and other conditions treatable by the encapsulated therapeutically active agent are also disclosed. Further disclosed are processes of preparing the casein particles. The disclosed casein particles are useful for orally delivering the therapeutically active encapsulate and can further be used for controllably releasing the agents in the gastrointestinal tract.

### FIELD AND BACKGROUND OF THE INVENTION

The present invention, in some embodiments thereof, relates to novel compositions and methods for treating cancer and more specifically, but not exclusively, to compositions for oral administration of chemotherapeutic agents and to uses thereof in the treatment of cancer. The present invention, in further embodiments thereof, relates to novel compositions and methods for treating medical conditions that are otherwise treatable by parenteral administration of therapeutically active agents and more specifically, but not exclusively, to compositions for oral administration of such therapeutically active agents and to uses thereof in the treatment of medical conditions treatable by these therapeutically active agents.

Beta-casein (β-CN), one of the four main caseins in bovine milk, is a protein that has a well defined hydrophilic N-terminal domain and a hydrophobic C-terminal domain. The pronounced amphiphilic structure of this protein imparts many properties that resemble those of low molecular weight surfactants. Similarly, beta-casein tends to self-associate under appropriate conditions, so as to form stable micelle-like nanoparticles in aqueous solution. The monomers (single β-CN molecules) in such micelle-like structure have a radius of gyration (Rg) of 4.6 nm, and the micelles, containing 15-60 molecules, have Rg values ranging between 7.3 and 13.5 nm. The critical micellization concentration (CMC) ranges between 0.05 and 0.2% w/v, depending on temperature, pH, solvent composition and ionic strength.

Several studies have investigated the binding of lipophilic molecules to β-CN, including vitamin D3, vitamin A, sucrose esters and sodium dodecyl sulfate. These studies suggested that hydrophobic interactions are largely responsible for the binding [Forrest et al. 2005 Journal of Agricultural and Food Chemistry 53[20]: 8003-8009].

Certain casein micelles are known in the art. U.S. Pat. No. 5,173,322 teaches the production of reformed casein micelles and the use of such micelles as a complete or partial replacement of fat in food product formulations. Related U.S. Pat. No. 5,318,793 teaches powdered coffee whitener containing reformed casein micelles. U.S. Pat. No. 5,833,953 teaches a process for the preparation of fluorinated casein micelles in which at least 100 ppm of a soluble fluoride salt are added to a solution comprising micellar casein.

U.S. Pat. No. 6,991,823 discloses a process for the preparation of mineral fortified milk comprising the addition of an amount of a pyrophosphate or orthophosphate to the milk in order to enable the mineral to migrate into the protein micelles.

U.S. Pat. No. 6,652,875 provides a formulation for the delivery of bioactive agents to biological surfaces comprising at least one isolated and purified casein protein or salt thereof in water. The invention relates to particular isolated and purified casein phosphoproteins in the form of casein calcium phosphate complexes intended to remain on the surface of oral cavity tissues or the gastrointestinal tract.

U.S. patent application Ser. No. 09/932,503, having Publication No. 2002/0054914, teaches an oral drug delivery system comprised of calcium phosphate particles, complexed with a therapeutic agent, and encased or enclosed by casein micelles, which can be used for orally delivering drugs such as insulin. The casein molecules taught by U.S. patent application Ser. No. 09/932,503 are arranged, presumably as micelles, around calcium phosphate particles containing the active drug, and are linked to the therapeutic agent-containing microparticles by mainly calcium phosphate and electrostatic bond interactions. According to the teachings of U.S. patent application Ser. No. 09/932,503, the resulting complex provides a carrier designed to protect the therapeutic agent in the harsh, acidic environment of the stomach before releasing therapeutic agent into the small intestine.

U.S. Pat. No. 6,503,545 teaches a composition for oral administration to humans and other mammals, comprising a mixture of at least one mammalian milk protein or fragment thereof, and at least one fat-soluble vitamin. An exemplary milk protein taught in this patent is casein, and the fat-soluble vitamin is Vitamin E. According to the teaching of U.S. Pat. No. 6,503,545, casein micelles and/or micelles formed with casein digestion fragments, can integrate vitamin E molecules and promote their absorption into the bloodstream.

WO 2007/122613, and the corresponding paper by some of the present inventors [Semo et al. 2007, Food Hydrocolloids, 21: 936-942], teaches isolated, re-assembled casein micelles which are useful for the encapsulation of hydrophobic biologically active agents such as Vitamin D and for delivering these agents in food and beverages.

The use of casein as a surface modifier, adsorbed on the surface of drug particles or drug containing particles, for enabling higher solubility, stability and reduced toxicity of the drug, have been disclosed in U.S. Pat. No. 5,399,363, and in U.S. patent applications having Publication Nos. 2008/0145432 and 2007/0166368.

U.S. patent application Ser. No. 10/652,814, having Publication No. 2004/0137071, teaches nanocapsules formed by partitioning a bioactive component (such as a drug) within a core of surfactant molecules, and surrounding the surfactant molecules with a biocompatible polymer shell. The biocompatible polymer taught by U.S. patent application Ser. No. 10/652,814 can be casein.

U.S. patent application Ser. No. 10/260,788, having Publication No. 2003/0180367, teaches a process of preparing stable microparticles of water-insoluble or poorly soluble compounds, which comprises mixing the particles of a water-insoluble or poorly soluble compound with at least one phospholipid and at least one surfactant to form a mixture, and applying energy to the mixture, sufficient to produce microparticles of the compound. The surfactant taught by U.S. patent application Ser. No. 10/260,788 can be casein.

Treatment of many diseases usually requires repeated subcutaneous injections of drugs. Such a mode of treatment causes discomfort and inconvenience to the patient. The oral delivery of such drugs is often precluded by acid digestion of the drugs in the stomach and digestion in the small intestine. This is particularly true with proteins and peptides, which are difficult or impossible to administer orally since they are easily digested or hydrolyzed by enzymes and/or other components present in gastric juices and other fluids secreted by the digestive tract. Injection is often the primary alternative administration method, but is unpleasant, expensive, and is not well tolerated by patients, particularly those requiring treatment for chronic illnesses.

Oral delivery of drugs is also often precluded by the poor solubility of the drug in aqueous solution. Poor water solubility of drugs is often associated with cancer treatment.

Currently available cancer treatments include surgery, chemotherapy, radiation therapy, and hormonal therapy, as well as immunotherapy.

The current chemotherapy approach suffers from a number of drawbacks. First, many chemotherapeutic drugs are hydrophobic and are thus hardly soluble in aqueous solutions. Therefore, most of the currently used chemotherapeutic drugs cannot be administered orally and are administered intravenously (IV). This route of administration is a major source of cost, discomfort and stress to patients, and multiple hospitalizations are required in order to complete the relatively long chemotherapeutic regimen. These devices are expensive, painful in the short term, and are associated with complications (such as, for example, infections and bleeding).

Secondly, conventional anti-cancer treatments such as radiation therapy or chemotherapy which is aimed at eradication of rapidly dividing cells, do not sufficiently discern between cancer cells and healthy tissues such as bone marrow cells and the gastrointestinal epithelial mucosa. Consequently, healthy cells are often damaged during drug treatment, resulting in toxic side effects.

### SUMMARY OF THE INVENTION

In view of the disadvantages associated with currently used methodologies for practicing cancer treatment with chemotherapeutic agents, methods for orally delivering chemotherapeutic agents are highly desirable. Methods for orally delivering other therapeutically active agents, particularly those used to treat chronic medical conditions are also highly desirable, in order to improve patient compliance.

The present inventors have now surprisingly uncovered that oral delivery of hydrophobic chemotherapeutic agents, which are typically administered intravenously, can be effected by utilizing micellar casein nanoparticles for encapsulating the drug. Such casein nanoparticles having a hydrophobic chemotherapeutic agent encapsulated therein have been successfully prepared and characterized, and were shown to encapsulate different hydrophobic drugs with high affinity association. The optimal encapsulation conditions were 1 mg/ml β-CN, ≦6% (v/v) DMSO in PBS. Under these conditions, particles of about 25-300 nm diameter were formed.

The present invention, in some embodiments thereof, is therefore of casein particles having encapsulated therein hydrophobic therapeutically active agent, which are particularly useful for orally delivering the active agent. The casein particles allow the lipid-soluble agent to be thermodynamically stable in aqueous solutions and to be readily delivered to the gastrointestinal tract (GIT). The casein particles can further comprise agents that are capable of directing the drug-containing particle to the required target zones along the GIT.

The casein particles can further act by themselves for targeting the active agent into the GIT lumen, due to the digestion thereof on in the GIT, which leads to release of the agent.

The casein particles are therefore highly useful as an oral delivery system of therapeutically active agents that are otherwise administered parenterally, and can be beneficially utilized for treating a variety of medical conditions, including cancer. The casein particles are particularly useful for orally delivering therapeutically active agent for treating gastric ailments.

According to an aspect of some embodiments of the invention there is provided a casein particle having encapsulated therein a therapeutically active agent, the therapeutically active agent being a chemotherapeutic agent.

In some embodiments of the invention, the chemotherapeutic agent is a hydrophobic chemotherapeutic agent.

In some embodiments of the invention, the chemotherapeutic agent is characterized by a value of Log P that ranges from 1 to 10.

In some embodiments of the invention, the chemotherapeutic agent is a water-insoluble chemotherapeutic agent.

In some embodiments of the invention, the chemotherapeutic agent is administered parenterally if non-encapsulated.

In some embodiments of the invention, the chemotherapeutic agent is not suitable for oral administration if non-encapsulated.

In some embodiments of the invention, a water solubility of the chemotherapeutic agent is lower than 1% w/v.

In some embodiments of the invention, the chemotherapeutic agent is selected from the group consisting of paclitaxel, docetaxel, sn-38, irinotecan, doxorubicin (neutral), fluorouracil, bortezomib, camptothecin, carmustine, cisplatin, dactinomycin, docetaxel, floxuridine, ifosfamide, irinotecan, letrozole, mitomycin c, mitoxantrone, oxaliplatin, plicamycin, teniposide, valrubicin, vinblastine, vincristine and combinations thereof.

In some embodiments of the invention, the chemotherapeutic agent is selected from the group consisting of mitoxantrone, vinblastine, docetaxel, paclitaxel, irinotecan.

According to another aspect of embodiments of the invention there is provided a casein particle having encapsulated therein a therapeutically active agent that is administered parenterally if non-encapsulated, the therapeutically active agent being a hydrophobic therapeutically active agent and/or a water-insoluble therapeutically active agent.

In some embodiments of the invention, the therapeutically active drug is not suitable for oral administration if non-encapsulated.

In some embodiments of the invention, a water solubility of the therapeutically active agent is lower than 1% w/v.

In some embodiments of the invention, the casein is selected from the group consisting of beta casein, kappa casein and alpha casein.

In some embodiments of the invention, the casein is β-casein.

In some embodiments of the invention, the casein particle is having an average diameter lower than 800 nm.

In some embodiments of the invention, the casein particle is having an average diameter lower than 100 nm.

In some embodiments of the invention, the casein particle is a form selected from the group consisting of a micelle, a clustered micelle, a micellar cluster, a microparticle, a nanoparticle, a cluster of microparticles, a cluster of nanoparticles, a microcluster, a nanocluster, an aggregate, a microaggregate, a nanoaggregate, a particulate, a microparticulate and a nanoparticulate.

In some embodiments of the invention, a molar ratio of the therapeutically active agent to β-casein monomers forming the micelle ranges from 1:1 to 20:1.

In some embodiments of the invention, the casein particle further comprises a targeting moiety being attached to a surface thereof.

In some embodiments of the invention, the casein particle further comprises at least one additional agent being encapsulated therein or attached to a surface thereof.

In some embodiments of the invention, the additional agent and the therapeutically active agent act additively or in synergy.

In some embodiments of the invention, the casein particle is being for orally delivering the therapeutically active agent.

According to an aspect of some embodiments of the invention there is provided a pharmaceutical composition comprising any of the casein particles described herein.

In some embodiments of the invention, the pharmaceutical composition further comprises a pharmaceutically acceptable carrier.

In some embodiments of the invention, the pharmaceutical composition comprising a plurality of the casein particles.

In some embodiments of the invention, the pharmaceutical composition is being formulated for oral administration.

In some embodiments of the invention, the pharmaceutical composition is being packaged in a packaging material and identified in print, in or on the packaging material, for use in the treatment of cancer.

In some embodiments of the invention, the pharmaceutical composition is being packaged in a packaging material and identified in print, in or on the packaging material, for use in the treatment of a medical condition treatable by the therapeutically active agent.

According to a further aspect of some embodiments of the invention there is provided a use of any of the casein particles described herein in the manufacture of a medicament for treating cancer.

In some embodiments of the invention, the medicament is formulated for oral administration.

According to an additional aspect of some embodiments of the invention there is provided a method of treating cancer, the method comprising administering to a subject in need thereof the pharmaceutical composition as described herein.

In some embodiments of the invention, the administering is effected orally.

The casein particles have encapsulated therein a chemotherapeutic agent.

According to yet an additional aspect of some embodiments of the invention there is provided a use of any of the casein particles described herein in the manufacture of a medicament for treating a medical condition treatable by the therapeutically active agent.

In some embodiments of the invention, the medicament is formulated for oral administration.

According to still an additional aspect of some embodiments of the invention there is provided a method of treating a medical condition treatable by the therapeutically active agent, the method comprising administering to a subject in need thereof the pharmaceutical composition as described herein.

In some embodiments of the invention, the administering is effected orally.

According to another aspect of some embodiments of the invention there is provided a process of preparing the casein particle as described herein, the process comprising adding a solution containing the therapeutically active agent and a solvent to an aqueous solution containing casein, thereby obtaining the casein micelle.

In some embodiments of the invention, the solvent in the solution containing the therapeutically active agent is a water-miscible organic solvent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a graph showing the mean Gaussian diameter (▪) and back scattered light intensity at 480 nm, relative to the intensity at zero mitoxantrone concentration (▴) of β-CN and mitoxantrone particles as a function of mitoxantrone: β-CN molar ratio at 1 mg/ml β-CN calculated using dynamic light scattering (DLS) and a Fluorolog 3-22 spectrofluoremeter.

FIG. 2 presents zeta potential measurements of mitoxantrone in PBS (▪), and mitoxantrone in 1 mg/ml β-CN solution (♦) as a function of mitoxantrone concentration (bottom axis) and molar ratio (top). Shown in the insert is the chemical structure of mitoxantrone.

FIG. 3 presents a graph showing the percentage of the initial Trp 143 emission as a function of total mitoxantrone concentration (upper axis) and total mitoxantrone: β-CN molar ratio (lower axis) at 1 mg/ml β-CN (excitation: 287 nm and emission: 332 nm) wherein the line represents the model fit.

FIG. 4 present a plot showing the mitoxantrone fluorescence intensity as a function of β-CN concentration, at 0.042 mM (0.0217 mg/ml) mitoxantrone (excitation: 609 nm and emission: 675 nm) wherein the line represents the model fit.

FIG. 5 presents a bar graph showing the particle diameter distribution of vinblastine-containing β-CN particles. Shown is the percentage of particles having a diameter in the range of 0-100 nm (blank), 100-200 nm (blue), 200-300 nm (green) and 300 nm and up (yellow), as a function of vinblastine:β-CN molar ratio, at 1 mg/ml β-CN.

FIG. 6 presents a plot showing percentage of the initial Trp 143 emission as a function of total vinblastine concentration (lower axis) and total vinblastine:β-CN molar ratio (upper axis) at 1 mg/ml β-CN (excitation: 287 nm and emission: 332 nm). Line represents the model fit.

FIG. 7 presents plots showing the emission spectrum of 1 mg/ml (42 μM) pure β-CN (thick solid line) vs. that of vinblastine encapsulated in 1 mg/ml β-CN at 4:1 vinblastine:β-CN molar ratio (fine dashed line).

FIG. 8 presents comparative plots showing the absorbance spectra of 168 μM pure docetaxel (fine dashed line), 42 μM (1 mg/ml) pure β-CN (fine solid line), 168 μM docetaxel encapsulated in 42 μM β-CN (4:1 docetaxel:β-CN molar ratio) (thick dashed line) vs. a mathematical summation plot of pure docetaxel+pure β-CN (thick solid line).

FIG. 9 presents the measured zeta potential of docetaxel in PBS (▪), and docetaxel in 1 mg/ml β-CN solution (♦) as a function of docetaxel concentration (top axis) and docetaxel:β-CN molar ratio (bottom axis).

FIG. 10 presents images of 504 μM docetaxel encapsulated within 1 mg/ml β-CN nanoparticles at 12:1 molar ratio (left) and 504 μM free docetaxel in PBS and 4.55% DMSO but without β-CN (right). The solubilizing effect of β-CN can be observed from the clear solution obtained when the drug is in solution together with β-CN as compared to without.

FIG. 11 presents the emission spectrum of 1 mg/ml (42 μM) pure β-CN (thick solid line) vs. that of paclitaxel encapsulated in 1 mg/ml β-CN at 4:1 total paclitaxel:β-CN molar ratio (fine dashed line).

FIG. 12 presents the absorbance spectra of 168 μM pure paclitaxel (fine dashed line), 42 μM (1 mg/ml) pure β-CN (fine solid line), 168 μM paclitaxel encapsulated in 42 μM β-CN (4:1 paclitaxel:β-CN molar ratio) (thick dashed line) vs. a mathematical summation plot of the pure paclitaxel+pure β-CN spectra (thick solid line).

FIG. 13 presents images of 84 μM paclitaxel encapsulated within 1 mg/ml β-CN nanoparticles at 2:1 molar ratio (left) and the same concentration of 84 μM paclitaxel in PBS and 0.8% DMSO but without β-CN (right). The solubilizing effect of β-CN can be observed form the clear solution obtained when the drug is in solution together with β-CN as compared to without.

FIG. 14 presents comparative plots showing the absorbance spectra of 168 μM pure Irinotecan (fine dashed line), 42 μM (1 mg/ml) pure β-CN (fine solid line), 168 μM irinotecan encapsulated in 42 μM β-CN (4:1 irinotecan:β-CN molar ratio) (thick dashed line) vs. a mathematical summation plot of the pure Irinotecan+pure β-CN spectra (thick solid line).

FIG. 15 presents the measured zeta potential of irinotecan in PBS (▪), and irinotecan in 1 mg/ml β-CN solution (♦) as a function of total irinotecan concentration (top axis) and total irinotecan: β-CN molar ratio (bottom axis).

FIG. 16 presents Mean Gaussian diameter of irinotecan-containing β-CN nanoparticles as a function of irinotecan:β-CN molar ratio at 1 mg/ml β-CN.

FIG. 17 presents images of 504 μM irinotecan encapsulated within 1 mg/ml β-CN nanoparticles at 12:1 molar ratio (left) and 504 μM irinotecan solubilized in PBS and 5.6% DMSO but without β-CN (right). The solubilizing effect of β-CN can be observed form the clear solution obtained when the drug is in solution together with β-CN as compared to without.

### DESCRIPTION OF EMBODIMENTS OF THE INVENTION

The present invention, in some embodiments thereof, relates to novel compositions and methods for treating cancer and more specifically, but not exclusively, to compositions for oral administration of chemotherapeutic agents and to uses thereof in the treatment of cancer. The present invention, in further embodiments to thereof, relates to novel compositions and methods for treating medical conditions that are otherwise treatable by parenteral administration of therapeutically active agents and more specifically, but not exclusively, to compositions for oral administration of such therapeutically active agents and to uses thereof in the treatment of medical conditions treatable by these therapeutically active agents.

The present inventors have devised a methodology for successfully encapsulating chemotherapeutic agents in nano-sized beta-casein particles. Using this methodology, beta-casein nanoparticles encapsulating different drugs have been prepared and characterized, and were further shown to associate to the drug with high affinity. Exemplary encapsulation conditions were 1 mg/ml β-CN, ≦6% (v/v) DMSO in PBS. Under these conditions, particles of about 25-300 nm diameter were formed. The gastric digestibility of β-CN suggests possible targeting to stomach tumors. The stability and controlled release of molecules encapsulated in beta-casein particles, as detailed hereinbelow, render these particles highly advantageous over low molecular weight surfactants.

These drug-containing nano-sized particles can serve as oral delivery systems of chemotherapeutic agents and other therapeutically active agents that are not suitable for oral administration, and thus circumvent the need for the inconvenient and cost-inefficient intravenous administration, which is currently used in most of the chemotherapy regimens, and further facilitate administration regimes of other therapeutically active agents that are typically administered by injection. These drug-containing particles can further serve for selectively delivering the active agents to the GIT, in cases where use thereof for treating gastric ailments is desired, as detailed hereinbelow.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As demonstrated in the Examples section that follows, the successful encapsulation of the hydrophobic chemotherapeutic agents Mitoxantrone (MX), Vinblastin, Docetaxel, Paclitaxel and Irinoteacan, within β-CN nanoparticles, has been demonstrated.

As further demonstrated in the Examples section that follows, the drug encapsulation has been studies and characterized, as a function of the drug:beta-casein molar ration and other parameters, using various measurements (e.g., zeta potential, fluorescence emission, visual inspection, etc.). The obtained results demonstrate that β-CN nanoparticles display a very good binding and encapsulation capacity for the tested hydrophobic chemotherapeutic agents, and thus may serve as a useful nano- or microscopic vehicle for the solubilization and delivery of hydrophobic drugs in aqueous drug preparations such as preparations for oral administration of the drug.

Thus, according to one aspect of embodiments of the present invention there is provided a casein particle having encapsulated therein a hydrophobic therapeutically active agent.

Without being bound by a particular theory, it is suggested that the water solubility of the hydrophobic therapeutically active agent in the casein particles described herein, is enhanced by encapsulating the agent within the core, hydrophobic portion of the particle while the hydrophilic portion of the particle is exposed to the water.

It is further suggested, that in some cases the encapsulated agent is better protected from low pH-related and enzymatic disintegration in the stomach, thereby enhancing its oral bioavailability. Thus, the encapsulation of chemotherapeutic agents in the herein described casein particles enables the oral administration of chemotherapeutic agents which heretofore have been administered mainly via a parenteral rout due to their low bioavailability when administered orally for reasons such as drug disintegration in the stomach and/or low water solubility of the drug.

In addition, in some cases, the naturally good digestibility of -casein in the stomach acts as a target-activated release mechanism, a feature which can be utilized for treating gastric, or upper intestinal illnesses and ailments, including tumors such as cancerous tumors.

Thus, according to some embodiments of the invention, the therapeutically active agent is hydrophobic and/or water insoluble.

Accordingly, in some embodiments, the therapeutically active agent is not suitable for oral administration if non-encapsulated.

According to embodiments of the invention, the therapeutically active agent is such that is otherwise, when non-encapsulated, administered parenterally, as defined herein.

As used herein, the term “hydrophobic” describes a characteristic of an agent that renders it poorly soluble in aqueous environment.

Hydrophobicity is typically defined by the partition coefficient (P) of the agent that represents the ratio of its concentrations in a hydrophobic solvent vs. an aqueous solution (e.g., water). Usually, hydrophobicity values are expressed as Log P.

In some embodiments, the therapeutically active agent is characterized by a Log P value in the range of 1 to 10. In some embodiments, it is characterized by a Log P value in the range of 2 to 6.

The phrase “water insoluble” describes a characteristic of an agent that reflects a poor solubility of the agent in aqueous environment. Typically, poorly water-soluble agents are characterized by a solubility in aqueous solutions that is lower than 10% w/v, lower than 5% w/v, lower than 3% w/v, lower than 2% w/v, lower than 1% w/v and sometimes even lower than 0.5% w/v, or lower than 0.1% w/v.

In some embodiments, the water solubility of the therapeutically active agent is lower than 1% w/v.

As used herein, “% w/v” describes a weight percentage of a component or an agent in the total volume of the solution. Thus, for example, 1% w/v describes 1 gram of an agent in a 100 ml solution.

The casein particles described herein therefore enable the oral administration of therapeutically active agents that are otherwise typically utilized via parenteral administration.

As used herein, the phrase “parenteral administration” encompasses any route of administration other than the digestive tract, and typically refers to injection-based route of administration. This phrase encompasses, for example, injecting a drug directly into a vein (intravenous), muscle (intramuscular), artery (intrarterial), abdominal cavity (intrperitoneal), heart (intracardiac) or into the fatty tissue beneath the skin (subcutaneous).

The term “therapeutically active agent” describes a compound or compounds which are used to treat or prevent any disease or undesirable medical condition or a manifestation thereof, which afflicts a subject.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

Exemplary therapeutically active agent of which encapsulation within the casein particles described herein is beneficial include, but are not limited to, a steroid, a drug to treat osteoporosis, a blood coagulation factor, an antibiotic, lipase, a beta-blocker, an anti-asthma agent, an antisense, an anti-inflammatory agent, an anti-viral agent, an anti-hypertensive agent, a cardiovascular agent, an anti-arrythmia drugs, a diuretic and an anti-clotting agent.

In some embodiments, the therapeutically active agent is a non-proteinacious agent.

In some embodiments, the therapeutically active agent is a small molecule, as opposed to macromolecule (e.g., polymeric compounds such as proteins, oligopeptides, oligonucleotides, etc.).

In some embodiments, the therapeutically active agent is selected such that it is capable of attractively interacting with casein.

In some embodiments, the therapeutically active agent is a chemotherapeutic agent.

The encapsulation of chemotherapeutic agents in the herein described casein particles enables the oral administration of chemotherapeutic agents which heretofore have been administered mainly via a parenteral route due to their low bioavailability when administered orally for reasons such as drug disintegration in the stomach and/or low water solubility of the drug.

Thus, according to another aspect of embodiments of the present invention there is provided a casein particle having encapsulated therein a therapeutically active agent, wherein the therapeutically active agent is a chemotherapeutic agent.

Accordingly, in some embodiments, the chemotherapeutic agent is administered parenterally if non-encapsulated. In some embodiments the chemotherapeutic agent is not suitable for oral administration if non-encapsulated.

The phrase “chemotherapeutic agent” describes any therapeutically active agent that directly or indirectly eradicates proliferating cells, cancer cells in particular, or directly or indirectly prohibits, inhibits, stops or reduces the proliferation of cancer cells. Chemotherapeutic agents include those that result in cell death and those that inhibit cell growth, proliferation and/or differentiation. Preferably, the chemotherapeutic agent is selectively toxic against certain types of cancer cells but does not affect or is less toxic against normal cells. The phrase “chemotherapeutic agent” encompasses any compound that mediates cancerous-cell death by any mechanism including, but not limited to, apoptosis, inhibition of metabolism or DNA synthesis, interference with cytoskeletal organization, destabilization or chemical modification of DNA, etc.

As used herein, the phrase “chemotherapeutic agent” encompasses any suitable chemotherapeutic agent, including small organic molecules, peptides, oligonucleotides and the like as well as radiotherapeutic agents such as, for example, those comprising radioactive iodine ¹³¹I and beta particle emitter ⁹⁰Y.

A partial listing of currently available chemotherapeutic agents according to class, and including diseases for which the agents are presently indicated, is provided as Table A below. Each of these exemplary chemotherapeutic agents can be used in the context of embodiments of the invention.

TABLE A
Chemotherapeutic Agents Useful in Neoplastic Disease¹

| | Type of | | |
|---|---|---|---|
| Class | Agent | Name | Disease² |
| Alkylating | Nitrogen | Mechlorethamine | Hodgkin's disease, non-Hodgkin's |
| Agents | Mustards | (HN₂) | lymphomas |
| | | Cyclophosphamide | Acute and chronic lymphocytic |
| | | Ifosfamide | leukemias, Hodgkin's disease, |
| | | | non-Hodgkin's lymphomas, |
| | | | multiple myeloma, |
| | | | neuroblastoma, breast, ovary, |
| | | | lung, Wilms' tumor, cervix, |
| | | | testis, soft-tissue sarcomas |
| | | Melphalan | Multiple myeloma, breast, ovary |
| | | Chlorambucil | Chronic lymphocytic leukemia, |
| | | | primary macroglobulinemia, |
| | | | Hodgkin's disease, non- |
| | | | Hodgkin's lymphomas |
| | | Estramustine | Prostate |
| | Ethylenimines | Hexamethyl- | Ovary |
| | and | melamine | |
| | Methylmelamines | Thiotepa | Bladder, breast, ovary |
| | Alkyl | Busulfan | Chronic granulocytic leukemia |
| | Sulfonates | | |
| | Nitrosoureas | Carmustine | Hodgkin's disease, non-Hodgkin's |
| | | | lymphomas, primary brain |
| | | | tumors, multiple myeloma, |
| | | | malignant melanoma |
| | | Lomustine | Hodgkin's disease, non-Hodgkin's |
| | | | lymphomas, primary brain |
| | | | tumors, small-cell lung |
| | | Semustine | Primary brain tumors, stomach, |
| | | | colon |
| | | Streptozocin | Malignant pancreatic |
| | | | insulinoma, |
| | | | malignant carcinoid |
| | Triazenes | Dacarbazine | Malignant melanoma, |
| | | | Hodgkin's |
| | | Procarbazine | disease, soft-tissue sarcomas |
| | | Aziridine | |
| Antimetabolites | Folic Acid | Methotrexate | Acute lymphocytic leukemia, |
| | Analogs | Trimetrexate | choriocarcinoma, mycosis |
| | | | fungoides, breast, head and |
| | | | neck, lung, osteogenic sarcoma |
| | Pyrimidine | Fluorouracil | Breast, colon, stomach, pancreas, |
| | Analogs | Floxuridine | ovary, head and neck, urinary |
| | | | bladder, premalignant skin |
| | | | lesions (topical) |
| | | Cytarabine | Acute granulocytic and acute |
| | Purine Analogs | Azacitidine | lymphocytic leukemias |
| | and Related | Mercaptopurine | Acute lymphocytic, acute |
| | Inhibitors | | granulocytic, and chronic |
| | | | granulocytic leukemias |
| | | Thioguanine | Acute granulocytic, acute |
| | | | lymphocytic, and chronic |
| | | | granulocytic leukemias |
| | | Pentostatin | Hairy cell leukemia, mycosis |
| | | | fungoides, chronic lymphocytic |
| | | | leukemia |
| | | Fludarabine | Chronic lymphocytic leukemia, |
| | | | Hodgkin's and non-Hodgkin's |
| | | | lymphomas, mycosis fungoides |
| Natural | Vinca Alkaloids | Vinblastine (VLB) | Hodgkin's disease, non-Hodgkin's |
| Products | | | lymphomas, breast, testis |
| | | Vincristine | Acute lymphocytic leukemia, |
| | | | neuroblastoma, Wilms' tumor, |
| | | | rhabdomyosarcoma, Hodgkin's |
| | | | disease, non-Hodgkin's |
| | | | lymphomas, small-cell lung |
| | | Vindesine | Vinca-resistant acute |
| | | | lymphocytic |
| | | | leukemia, chronic myelocytic |
| | | | leukemia, melanoma, |
| | | | lymphomas, |
| | | | breast |
| | Epipodophyl- | Etoposide | Testis, small-cell lung and other |
| | Lotoxins | Teniposide | lung, breast, Hodgkin's |
| | | | disease, non-Hodgkin's |
| | | | lymphomas, acute granulocytic |
| | | | leukemia, Kaposi's sarcoma |
| | Antibiotics | Dactinomycin | Choriocarcinoma, Wilms' |
| | | | tumor, |
| | | | rhabdomyosarcoma, testis, |
| | | | Kaposi's sarcoma |
| | | Daunorubicin | Acute granulocytic and acute |
| | | | lymphocytic leukemias |
| | | Doxorubicin | Soft-tissue, osteogenic, and |
| | | 4′- | other sarcomas; Hodgkin's |
| | | Deoxydoxorubicin | disease, non-Hodgkin's |
| | | | lymphomas, acute leukemias, |
| | | | breast, genitourinary, thyroid, |
| | | | lung, stomach, neuroblastoma |
| | | Bleomycin | Testis, head and neck, skin, |
| | | | esophagus, lung, and |
| | | | genitourinary tract; |
| | | | Hodgkin's disease, non- |
| | | | Hodgkin's lymphomas |
| | | Plicamycin | Testis, malignant hypercalcemia |
| | | Mitomycin | Stomach, cervix, colon, breast, |
| | | | pancreas, bladder, head and |
| | | | neck |
| | Enzymes | L-Asparaginase | Acute lymphocytic leukemia |
| | Taxanes | Docetaxel | Breast, ovarian |
| | | Paclitaxel | |
| | Biological | Interferon Alfa | Hairy cell leukemia, Kaposi's |
| | Response | | sarcoma, melanoma, carcinoid, |
| | Modifiers | | renal cell, ovary, bladder, |
| | | | non-Hodgkin's lymphomas, |
| | | | mycosis fungoides, multiple |
| | | | myeloma, chronic granulocytic |
| | | | leukemia |
| | | Tumor Necrosis | Investigational |
| | | Factor | |
| | | Tumor- | Investigational |
| | | Infiltrating | |
| | | Lymphocytes | |
| Miscellaneous | Platinum | Cisplatin | Testis, ovary, bladder, head and |
| Agents | Coordination | Carboplatin | neck, lung, thyroid, cervix, |
| | Complexes | | endometrium, neuroblastoma, |
| | | | osteogenic sarcoma |
| | Anthracenedione | Mitoxantrone | Acute granulocytic leukemia, |
| | | | breast |
| | Substituted | Hydroxyurea | Chronicgranulocytic leukemia, |
| | Urea | | polycythemia vera, essential |
| | | | thrombocytosis, malignant |
| | | | melanoma |
| | Methyl | Procarbazine | Hodgkin's disease |
| | Hydrazine | | |
| | Derivative | | |
| | Adrenocortical | Mitotane | Adrenal cortex |
| | Suppressant | Aminoglutethimide | Breast |
| Hormones and | | | Acute and chronic lymphocytic |
| Antagonists | costeroids | | leukemias, non-Hodgkin's |
| | | | lymphomas, Hodgkin's disease, |
| | | | breast |
| | Progestins | Hydroxy- | Endometrium, breast |
| | | progesterone | |
| | | caproate | |
| | | Medroxy- | |
| | | progesterone | |
| | | acetate | |
| | | Megestrol acetate | |
| | Estrogens | Diethylstil- | Breast, prostate |
| | | bestrol | |
| | | Ethinyl estradiol | |
| | Antiestrogen | Tamoxifen | |
| | Androgens | Testosterone | |
| | | propionate | |
| | | Fluoxymesterone | |
| | Antiandrogen | Flutamide | Prostate |
| | Gonadotropin- | Leuprolide | Prostate, Estrogen-receptor- |
| | Releasing | Goserelin | positive breast |
| | hormone | | |
| | analog | | |

¹Adapted from Calabresi, P., and B. A. Chabner, “Chemotherapy of Neoplastic Diseases” Section XII, pp 1202-1263 in: Goodman and Gilman's The Pharmacological Basis of Therapeutics, Eighth ed., 1990 Pergamin Press, Inc.; and Barrows, L. R., “Antineoplastic and Immunoactive Drugs”, Chapter 75, pp 1236-1262, in: Remington: The Science and Practice of Pharmacy, Mack Publishing Co. Easton, PA, 1995.; both references are incorporated by reference herein, in particular for treatment protocols.
²Neoplasms are carcinomas unless otherwise indicated.

The enhancement of water solubility of chemotherapeutic agents, by encapsulation in the herein described casein particles, is especially beneficial due to the large group of chemotherapeutic agents which are hydrophobic and exhibit poor solubility in aqueous solution, thus rendering their oral administration problematic.

Thus, in some embodiments, the chemotherapeutic agent is a hydrophobic chemotherapeutic agent, as defined herein.

In some embodiments, the water solubility of the chemotherapeutic agent is lower than 1% w/v.

Exemplary chemotherapeutic agents that are characterized by pronounced poor water solubility and hence encapsulation thereof within the casein particles, described herein, is beneficial, include, but are not limited to, paclitaxel, docetaxel, sn-38, irinotecan, doxorubicin (neutral), abarelix, aldesleukin, alemtuzumab, asparaginase, azacitidine, bevacuzimab, fluorouracil, bleomycin, bortezomib, camptothecin, carmustine, cetuximab, cisplatin, dactinomycin, docetaxel, doxorubicin hydrochloride, floxuridine, fulvestrant, gemtuzumab, ibritumomab, ifosfamide, interferon alfa-2a, interferon alfa-2b, irinotecan, letrozole, leuprolide acetate, mitomycin c, mitoxantrone, oxaliplatin, plicamycin, rituximab, teniposide, tositumomab, trastuzumab, valrubicin, vinblastine, vincristine and combinations thereof.

In some embodiments, the chemotherapeutic agent is selected from the group consisting of Paclitaxel, Docetaxel, SN-38, irinotecan, doxorubicin (neutral), Fluorouracil, Bortezomib, Camptothecin, Carmustine, Cisplatin, Dactinomycin, Docetaxel, floxuridine, Ifosfamide, Irinotecan, Letrozole, Mitomycin C, Mitoxantrone, Oxaliplatin, Plicamycin, Teniposide, Valrubicin, Vinblastine, Vincristine and combinations thereof.

In some embodiments, the chemotherapeutic agent is any pharmaceutically acceptable derivative, salt, prodrug, analog, isomer, stereoisomer, isomorph or any other family member of any of the currently available chemotherapeutic agents, such as those mentioned herein.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. In some embodiments, the chemotherapeutic agent is Mitoxantrone or a pharmaceutically acceptable salt thereof.

Mitoxantrone (MX; Novantrone®) is a hydrophobic chemotherapeutic drug which inhibits type II topoisomerase inhibitor and disrupts DNA synthesis and DNA repair in both healthy cells and cancer cells. Mitoxantrone is made available to the target tissues by transport protein human serum albumin (HSA). The drug has an endogenous blue color, is poorly water soluble, has Log P higher than 2 and is administered via intravenous infusion.

As discussed hereinabove, the successful encapsulation of the hydrophobic chemotherapeutic agent Mitoxantrone by β-CN nanoparticles is presented in the Examples section that follows. The addition of Mitoxantrone at various MX:β-CN molar ratios resulted in the construction of MX encapsulated β-casein nanoparticles of various sizes including very small nanoparticles having an average diameter lower than 100 nm.

As discussed hereinabove and is further demonstrated in the Examples section that follows, the efficient encapsulation of Irinotecan, of taxanes such as Paclitaxel and Docetaxel, as well as of Vinblastine has also been successfully performed.

Therefore, in some embodiments, the chemotherapeutic agent is Irinotecan.

In some embodiments the chemotherapeutic agent is a taxane.

In some embodiments the chemotherapeutic agent is Paclitaxel.

In some embodiments the chemotherapeutic agent is Docetaxel.

In some embodiments the chemotherapeutic agent is Vinblastine.

In some embodiments, the chemotherapeutic agent is an analog, derivative, prodrug, salt, isomer, isomorph or any other family member of the above-mentioned drugs.

Irinotecan is a chemotherapeutic agent that targets Topoisomerase I and thus induces single strand breaks, thereby blocking cellular DNA replication.

Taxanes (e.g., paclitaxel, Docetaxel) are chemotherapeutic agents that bind to a β-tubulin and thereby form stable, non-functional microtubules and thus interfere with mitosis as well as with multiple cellular processes that require intact cytoskeleton. The Taxanes can also induce apoptosis and have anti-angiogenic properties.

Vinblastine is a chemotherapeutic agent that binds to β-tubulin as do taxanes, but in contradistinctions to the latter, vinblastine inhibits tubulin polymerization, and hence by blocking microtubule formation it blocks mitosis and thereby leads to cell death via apoptosis.

As discussed herein, any of the therapeutically active agents described herein are encapsulated within the casein particles.

The term “encapsulated” and its grammatical diversions, as used herein, describe a therapeutically active agent that is enclosed, enveloped, encased or entrapped within the casein particle, such that it is surrounded, partially or completely, by casein monomers that form the casein particle.

The term “casein” describes the predominant phosphoprotein in non-human mammals milk, which comprises the subgroups (also referred to hereinbelow as monomers) α_{S1}, α_{S2}, β (beta) and κ (kappa).

Accordingly, in some embodiments, the casein particles described herein are formed from casein monomers, whereby the casein monomers can be one or more of beta casein, kappa casein and alpha casein.

The casein particles described herein are formed from a solution containing the respective casein monomer(s), as opposed to casein particles that are formed by re-assembling naturally-occurring casein micelles.

In some embodiments, the casein particles described herein are formed from beta-casein (also referred to herein β-casein or β-CN) monomers. Such particles are also referred to herein, interchangeably, as beta-casein particles or micelles, β-casein particles or micelles, and β-CN particles or micelles. The terms particles or micelles may also be described by such terms as microparticles, nanoparticles, assemblies, or self-assemblies, clusters, nanoclusters, aggregates, nanovehicles, particulates, and the like.

β-casein (β-CN), one of the four main caseins, is a protein that has a well defined hydrophilic N-terminal domain and a hydrophobic C-terminal domain, which renders it highly suitable in the context of embodiments of the invention.

Moreover, it has recently been suggested that the amphiphilic structure of β-CN is analogous to that of an amphiphilic diblock copolymer, since both share some aspects of behavior in solution, such as the formation of micellar aggregates [Horne, D. S. Current Opinion in Colloid & Interface Science, 2002, 7, 456-461]. In β-CN, the hydrophobic regions interact intermolecularly in solution, rather than compact themselves into a folded globular form, as in the case of low molecular weight surfactants. As opposed to such surfactants, block co-polymers, and so β-CN, are highly stable, and the kinetics of release of hydrophobic molecules entrapped therein are several orders of magnitude (e.g., 7 or 8) slower compared to release from low molecular weight surfactants. Moreover, the relatively low CMC of block-copolymers assures that they are not likely break apart spontaneously, to thereby uncontrollably release molecules entrapped thereby, as is often the case with low MW surfactants [Zana, R. In Dynamics of Surfactant Self-Assemblies, Zana, R. Ed. CRC Press, Taylor & Francis Group: New York, 2005; pp. 161-231].

Thus, the relative stability and controlled-release attributed to beta-casein particles is highly advantageous in the context of some embodiments of the invention.

The term “particle”, as used herein, describes an assembly of casein monomers, which is typically formed in solution so as to minimize the contact between the lyophobic (“solvent-repelling”) portion of the casein molecule and the solvent. Such an assembly includes, for example, aggregation of casein monomers into structures such as spheres, cylinders or sheets, wherein the lyophobic portions are on the interior of the aggregate structure and the lyophilic (“solvent-attracting”) portions are on the exterior of the structure.

The assembled casein monomers can therefore form closed structures, partially closed structures and/or open structures.

The particle size of casein assembled structures is typically in the range of nano-sized particles to micro-sized particles.

Thus, the term “particle” encompasses microparticles and nanoparticles. The term “β-casein nanoparticles” describes nanoparticles formed from β-casein monomers.

The term “particle” further encompasses micelles, clustered micelles, a micellar cluster, a microparticle, a nanoparticle, a cluster of microparticles, a cluster of nanoparticles, a microcluster, a nanocluster, an aggregate, a microaggregate, a nanoaggregate, a particulate, a microparticulate, and a nanoparticulate.

The term “cluster”, as used herein, describes an ensemble of particles that are bound or interacted with one another so as to form a larger particle. A micellar cluster therefore describes a cluster formed from several micelles, a cluster of nanoparticles or microparticles describes a cluster formed from several microparticles or nanoparticles, respectively. A microcluster describes a micro-sized cluster. A nanocluster describes a nano-sized cluster.

The term “aggregate” describes a particle formed from assembled components. Microaggregate and nanoaggregates describe micro-sized and nano-sized aggregate, respectively.

The term “particulate” describes a plurality of individually dispersed particles.

In some embodiments, the casein particles are in the form micelles.

As used herein, the term “micelle” describes a colloidal particle, in a simple arrangement or geometric form, typically spherical, of a specific number of amphipathic molecules, which forms at a well-defined concentration, called the critical micelle concentration. The micelle can be a single particle or can form a cluster of several micelles, which interact with one another so as to form a particle.

Thus, is some embodiments, the casein particles described herein are in the form of micelles or clustered micelles.

As discussed hereinabove, the successful encapsulation of the hydrophobic chemotherapeutic agents in β-casein nanoparticles has been achieved and is presented in the Examples section that follows.

Accordingly, in some embodiments, the casein particle is a nanoparticle, such as a β-casein nanoparticle.

Thus, in some embodiments, the casein particles have an average diameter that ranges from 1 nm to 1000 nm. In some embodiments, the casein particles have an average diameter lower than 1000 nm, lower than 900 nm, lower than 800 nm, lower than 700 nm, lower than 600 nm, lower than 500 nm, lower than 400 nm, lower than 300 nm, lower than 200 nm. In some embodiments, the average diameter is lower than 100 nm.

When the casein nanoparticles are clustered micelles, the particles can have larger average diameter, for example, in the range of from 500 nm to 2 microns.

In some embodiments of the invention, the casein particle is such that the molar ratio of the therapeutically active agent to casein monomers forming the particle ranges from 1:1 to 20:1.

In some embodiments, this molar ratio ranges from 1:1 to 10:1, from 1:1 to 8:1, and in some embodiments, this ratio ranges from 1:1 to 6:1.

In some embodiments the molar ratio of the therapeutically active agent to β-casein monomers forming the particles ranges from 2.2:1 to 3.3:1.

As further presented in the Examples section that follows, the stoichiometry of the binding of Mitoxantrone to the β-CN nanoparticles was determined using dynamic light scattering (DLS), scattered light intensity and fluorescence emission studies. The calculated values of Mitoxantrone: β-CN molar ratio within the β-CN nanoparticle system was between 2.2-3.3 moles of Mitoxantrone per mole of protein (see, Table 1) with the Mitoxantrone loading per β-CN being in the range of about 90-360 molecules MX per β-CN nanoparticle. The casein particles described herein enable the hydrophobic therapeutically active agent (e.g., chemotherapeutic agents), encapsulated within the particles, to be thermodynamically stable in aqueous solutions and to be readily delivered to the gastrointestinal tract (GIT). Without being bound by any particular theory, it is suggested that the release of the agent from the particle in the GIT is achieved mainly due to the casein particle disintegration, as a result of contacting GIT hydrolases, which results in the release of the entrapped agent into the GIT lumen, particularly, the stomach.

The addition of a targeting moiety onto the surface of the casein particle can direct the therapeutically active agent to a specific target site in the GIT. For example, the addition of a targeting moiety being an antibody against a specific tumor-related antigen may enable the specific delivery of the chemotherapeutic agent to the tumor site.

Thus, according to some embodiments, the casein particles described herein further comprise a targeting moiety being attached to as surface thereof. Exemplary targeting moieties include, but are not limited to, antibodies, receptor ligands, enzyme substrates and the like.

In some embodiments, the casein particles further comprise at least one additional agent being encapsulated therein or attached to the surface thereof.

The additional agent can be such that enhances the therapeutic activity of the therapeutically active agent encapsulated in the casein particle or of the particle described herein as whole.

The agent may be, for example, an additional therapeutically active agent. In some embodiments, the additional therapeutically active agent is selected such that it acts in synergy with the therapeutically active agent described herein, so as to exhibit a synergistic therapeutic activity. For example, the additional agent can be a chemotherapeutic agent that acts in synergy with the chemotherapeutic agent encapsulated in the casein particle. Alternatively, the additional agent may be a compound which stabilizes the particle by enhancing the electrostatic and/or covalent bonding between the casein monomers (for example a salt or a crosslinker).

In some embodiments, the additional agent is selected from the group consisting of Paclitaxel, Docetaxel, SN-38, irinotecan, doxorubicin (neutral), Fluorouracil, Bortezomib, Camptothecin, Carmustine, Cisplatin, Dactinomycin, Docetaxel, floxuridine, Ifosfamide, Interferon Alfa-2a, Interferon Alfa-2b, Irinotecan, Letrozole, Mitomycin C, Mitoxantrone, Oxaliplatin, Plicamycin, Teniposide, Valrubicin, Vinblastine, Vincristine and combinations thereof.

As discussed hereinabove and is exemplified in the Examples section that follows, the successful encapsulation of the hydrophobic chemotherapeutic agents in β-casein nanoparticles has been achieved. Thus the casein particles described herein may serve as a useful nanoscopic vehicle for the solubilization and delivery of hydrophobic drugs in aqueous drug preparations.

As further discussed hereinabove, oral administration of hydrophobic drugs, for the treatment of various disorders and disease conditions is often precluded by the poor solubility of the drug in aqueous solution. The encapsulation of a hydrophobic drug within the casein particle described herein enables the oral administration of the drug, for treating a disorder or disease condition that is typically treatable by, for example, parenteral administration of the drug.

Thus, according to another aspect of embodiments of the present invention, there is provided a method of treating a medical condition treatable by the therapeutically active agent. The method is effected by administering to a subject in need thereof the casein particles described herein. In some embodiments, the administering is effected orally.

According to another aspect of embodiments of the present invention, there is provided a use of the casein particle described herein in the manufacture of a medicament for treating a medical condition treatable by the therapeutically active agent. In some embodiments the medicament is formulated for oral administration.

As discussed hereinabove, a large group of chemotherapeutic agents are hydrophobic and exhibit poor solubility in aqua solution, thus rendering their oral administration problematic.

Many chemotherapeutic agents are typically administered intravenously (IV). This route of administration is a major source of cost, discomfort and stress to patients, and multiple hospitalizations are required in order to complete the relatively long chemotherapeutic regimen.

Thus, the enhancement of chemotherapeutic agent's solubility, by encapsulation in the herein described casein particles is especially beneficial and may be utilized for treating cancer and cancer metastases.

The terms “cancer” and “tumor” are used interchangeably herein to describe a class of diseases in which a group of cells display uncontrolled growth (division beyond the normal limits). The term “cancer” encompasses malignant and benign tumors as well as disease conditions evolving from primary or secondary tumors. The term “malignant tumor” describes a tumor which is not self-limited in its growth, is capable of invading into adjacent tissues, and may be capable of spreading to distant tissues (metastasizing). The term “benign tumor” describes a tumor which is not malignant (i.e. does not grow in an unlimited, aggressive manner, does not invade surrounding tissues, and does not metastasize). The term “primary tumor” describes a tumor that is at the original site where it first arose and the term “secondary tumor” describes a tumor that has spread from its original (primary) site of growth to another site, close to or distant from the primary site.

Cancers treatable with the present invention include but are not limited to solid, including carcinomas, and non-solid, including hematologic, malignancies. Carcinomas include, but are not limited to, adenocarcinomas and epithelial carcinomas. Hematologic malignancies include, but are not limited to, leukemias, lymphomas, and multiple myelomas. The following are non-limiting examples of the cancers treatable with the casein particles described herein: ovarian, colon, rectal, colorectal, melanoma, lung, breast, kidney, and prostate cancers. In some embodiments the cancer is located in the GIT. In some embodiments, the cancer is selected from the group consisting of colon cancer, rectal cancer and colorectal cancer.

The term “cancer metastases” describes cancer cells which have “broken away”, “leaked”, or “spilled” from a primary tumor, entered the lymphatic and/or blood vessels, circulated through the lymphatic system and/or bloodstream, settled down and proliferated within normal tissues elsewhere in the body, thereby creating a secondary tumor. In some embodiments, the cancer metastases treatable with the present invention are located in the GIT.

Accordingly, according to another aspect of embodiments of the invention, there is provided a method of treating cancer, the method comprising administering to a subject in need thereof casein particles as described herein, encapsulating a chemotherapeutic agent, as described herein.

In some embodiments, the administering is effected orally.

According to another aspect of embodiments of the present invention, there is provided a use of the casein particles described herein, encapsulating a chemotherapeutic agent, as described herein, in the manufacture of a medicament for treating cancer. In some embodiments the medicament is formulated for oral administration.

In any of the methods and uses described herein, the casein particle described herein can be utilized either per se or being formulated into a pharmaceutical composition which may further comprise a pharmaceutically acceptable carrier.

Thus, according to another aspect of embodiments of the invention there is provided a pharmaceutical composition which comprises a casein particle, as described herein.

As used herein a “pharmaceutical composition” refers to a preparation of one or more of the casein particles described herein, with other chemical components such as pharmaceutically acceptable and suitable carriers and excipients. The purpose of a pharmaceutical composition is to facilitate administration of a compound to an organism. Herein, the phrase “pharmaceutically acceptable carrier” describes a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

In some embodiments the pharmaceutical composition is formulated for oral administration.

In some embodiments the pharmaceutical composition comprises a plurality of the casein particles.

Pharmaceutical compositions as described herein may be manufactured by processes well known in the art, e.g., by means of conventional mixing, dissolving, granulating, dragee-making, levigating, emulsifying, encapsulating, entrapping or lyophilizing processes.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with embodiments of the invention thus may be formulated in conventional manner using one or more pharmaceutically acceptable carriers comprising excipients and auxiliaries, which facilitate processing of the particles described herein into preparations which, can be used pharmaceutically. Proper formulation is dependent upon the route of administration chosen. In some embodiments the pharmaceutical composition is formulated for oral administration.

According to some embodiments, the pharmaceutical composition is formulated as a solution, suspension or emulsion.

According to some embodiments, the pharmaceutical composition further includes a formulating agent selected from the group consisting of a suspending agent, a stabilizing agent and a dispersing agent.

For injection, the casein particles described herein may be formulated in aqueous solutions, preferably in physiologically compatible buffers such as Hank's solution, Ringer's solution, or physiological saline buffer with or without organic solvents such as propylene glycol, or polyethylene glycol.

For transmucosal administration, penetrants are used in the formulation. Such penetrants are generally known in the art.

In some embodiments, the pharmaceutical composition, comprising the casein particles described herein, is formulated for oral administration.

The casein particles described herein may be in the form of a solid such as a powder, tablet, pill, dragees capsules and the like, in which case the particles are dispersed upon physical contact with the physiological liquids located in the GIT. For oral administration, the casein particles described herein can be formulated readily by combining the particles with pharmaceutically acceptable carriers well known in the art. Such carriers enable the particles described herein to be formulated as tablets, pills, dragees, capsules, liquids, gels, syrups, slurries, suspensions, and the like, for oral ingestion by a patient. In some embodiments the pharmaceutical compositions for oral administration include aqueous solutions or aqueous suspensions of the casein particles described herein in water-soluble form. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active agent doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the agent(s) may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the casein particles described herein are conveniently delivered in the form of an aerosol spray presentation (which typically includes powdered, liquified and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the casein micelle and a suitable powder base such as, but not limited to, lactose or starch.

The casein particles described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the casein particles described herein in water-soluble form. Additionally, suspensions of the casein particles may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the agents to allow for the preparation of highly concentrated solutions.

Alternatively, the casein particles may be in powder form for constitution with a suitable vehicle, e.g., sterile, pyrogen-free water, before use.

The casein particles described herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of an agent as described herein effective to prevent, alleviate or ameliorate symptoms of a physiological disorder associated with cancer (such as stomach cancer) or prolong the survival of the subject being treated.

Determination of a therapeutically effective amount is well within the capability of those skilled in the art, especially in light of the detailed disclosure provided herein.

For any of the casein particles utilized in the methods and uses of the invention, the therapeutically effective amount or dose can be estimated initially from activity assays in animals. For example, a dose can be formulated in animal models to achieve a circulating concentration range that includes the IC₅₀ as determined by activity assays (e.g., when the encapsulated agent within the casein particle is a chemotherapeutic agent, the IC₅₀ may be the concentration of the particles, which achieves a 50% reduction in tumor size upon administration of the casein particles to an animal suffering from the tumor). Such information can be used to more accurately determine useful doses in humans.

Toxicity and therapeutic efficacy of the casein particles described herein can be determined by standard pharmaceutical procedures in experimental animals, e.g., by determining the EC₅₀, the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present embodiments may, if desired, be presented in a pack or dispenser device, such as an FDA (the U.S. Food and Drug Administration) approved kit, which may contain one or more unit dosage forms containing the active agent. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising casein particles as described herein, formulated in a compatible pharmaceutical carrier may also be prepared, placed in an appropriate container, and labeled for treatment of an indicated condition, as is detailed herein.

The pharmaceutical composition may be formulated so that the casein to monomers and therapeutically active agent are separately packed and are mixed together immediately prior to administration, in which time the particles are formed and the agent is encapsulated within the particle. Alternatively, the pharmaceutical composition may be formulated so that the therapeutically active agent is already encapsulated within the casein particle and is ready for administration to a subject in need thereof.

Thus, according to an embodiment of the present invention, the pharmaceutical composition is packaged in a packaging material and identified in print, in or on the packaging material, for use in the treatment of a medical condition treatable by the therapeutically active agent encapsulated within the casein particle, as detailed herein.

According to another embodiment, the pharmaceutical composition is packaged in a packaging material and identified in print, in or on the packaging material, for use in the treatment of cancer, as described herein.

In any of the methods, used and compositions described herein, the casein particles can be utilized in combination with an additional active agent, as described hereinabove. Such an additional active agent can form a part of the casein particle, as described hereinabove, or can be co-administered with the casein particle.

In some embodiments, such an additional agent can be co-formulated with the casein particles in a pharmaceutical composition as described herein.

According to another aspect of embodiments of the present invention, there is provided a process of preparing the casein particles described herein. The process is generally effected by adding a solution containing the therapeutically active agent and a solvent to an aqueous solution containing casein, thereby obtaining the casein particle.

In some embodiments, the solvent in the solution containing the therapeutically active agent is an organic solvent.

In some embodiments, the casein monomer is β-casein (β-CN). In some embodiments the aqueous solution is sodium phosphate buffer solution (PBS) having a pH of 7.0 and ionic strength of 0.1.

In some embodiments, the therapeutically active agent is a chemotherapeutic agent.

The concentration of the casein in the aqueous solution can be above, below or at its critical micelle concentration.

The phrase “critical micelle concentration” (CMC) describes the concentration of casein monomer above which the casein monomers are present substantially in a micellar form under a given set of conditions. At the vicinity of CMC, sharp change in many experimental parameters may be observed, and this may be measured by a number of techniques that include, but not limited to, surface tension measurements, fluorescence, conductivity, osmotic pressure, and the like. CMC varies as a function of a number of physical factors such as pH, temperature and pressure.

In some embodiments the process described herein, is such wherein the concentration of the therapeutically active agent in the solution and the concentration of the casein in the aqueous solution is selected so as to obtain a pre-determined molar ratio of the therapeutically active agent to casein monomers forming the particle. Exemplary molar ratios are described hereinabove.

The construction of the desired casein particles, comprising the encapsulated therapeutically active agent may be verified by techniques well known in the art. Examples of such techniques are detailed in the Examples section that follows and include, zeta potential measurements, DLS, scattered light intensity, and fluorescence techniques.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

### Materials and Methods

Materials:

Docetaxel trihydrate (purity 99.52%, HPLC) and irinotecan hydrochloride trihydrate (purity 99.75%, HPLC) were purchased from Iffect Chemphar Co. LTD. P.R. China.

Mitoxantrone dihydrochloride (M6545, purity>97% HPLC), vinblastine sulfate salt (V1377, purity≧96%, HPLC), paclitaxel (T7191 from semisynthetic (from Taxus sp.), ≧97%), and β-CN from bovine milk (C6905, purity 90%) were purchased from Sigma-Aldrich Israel Ltd. and used without further purification.

Stock solutions of about 10 mM drug in dimethyl sulfoxide (DMSO) were prepared. β-CN was dissolved in sodium phosphate buffer solution (PBS) pH 7.0, ionic strength 0.1, at different concentrations. The PBS solution was composed of 80 mM NaCl, 5.65 mM Na₂HPO₄ and 3.05 mM NaH₂PO₄.

The encapsulation of each drug in β-CN nanoparticles at different drug:β-CN molar ratios, was performed by titration of different volumes of drug in DMSO solution to the β-CN solution in PBS while stirring. The volume percentage of DMSO in PBS did not exceed 6%. The samples were equilibrated overnight at room temperature.

Nanoparticle Size Distribution and Zeta Potential Analysis:

Particle size distribution, mean Gaussian diameter, and zeta potential were determined by a combined dynamic light scattering (DLS) and zeta potential analyzer (NICOMP 380, Particle Sizing Systems (Agilent Technologies, Inc.), Santa Barbara, Calif., USA) at 25° C.

The effects of the drug: β-CN molar ratio on the mean Gaussian particle diameter, as well as on the particle size distribution, were determined. Solutions of different drug:β-CN molar ratio, from 0.2:1 and up to 12:1, were prepared by adding different volumes of drug in DMSO to 1 mg/ml β-CN in PBS.

Zeta potential was measured in PBS solutions without NaCl, under a 3 V/cm e-field, using phase analysis mode. The zeta potential was calculated from the electrophoretic mobility (EM) using the Smoluchowski model (which is a reasonable approximation given that the radius of the particles (a) was around 125 nm, and the ionic strength during the EM measurement was calculated to be about 50 mM, i.e. the Debye length (κ⁻¹) was around 1.4 nm, so that the product κ⁻¹a=92>>1).

Back Scattered Light Intensity:

Back Scattered light intensity measurements of drug encapsulated in β-CN at different drug:β-CN molar ratios were performed, using a spectrofluoremeter (Fluorolog 3-22, Jobin Yvon, Horiba, Longjumeau cedex, France) at a front-face mode. To study elastic light-scattering, the excitation and emission wavelengths were both set at 480 nm, (a wavelength at which both drug and β-CN have minimal absorbance), using slit widths of 1 nm.

Absorbance Spectra Analysis:

The absorbance spectra of (i) the tested drugs alone, at the indicated concentration, (ii) β-CN alone (1 mg/ml, corresponding to 0.042 mM), and (iii) β-CN encapsulated drugs at the indicated total drug:β-CN molar ratio (β-CN at a concentration of 1 mg/ml), were collected using an Ultrospec 3000 spectrophotometer (GE Healthcare). The absorbance was calibrated using a PBS and DMSO blank.

Tryptophan (Trp) Fluorescence:

Trp 143 is located in the main hydrophobic domain of β-CN. Quenching of protein fluorescence due to energy transfer from this Trp residue to a bound ligand serves to determine the binding affinity.

β-CN-drug interaction was studied by monitoring the changes in the Trp fluorescence emission of β-CN upon addition of the drug. Trp fluorescence was determined using an excitation at 287 nm and emission was detected at 332 nm, with slit widths of 1 nm, using the Fluorolog 3-22 spectrofluoreineter. Intrinsic fluorescence of the Trp residues of β-CN was measured before and after addition of different amounts of each drug ranging between drug: β-CN molar ratio of 0.2:1 to 12:1 at 1 mg/ml β-CN. Changes in Trp fluorescence were used to evaluate the binding of the drug to β-CN. The apparent dissociation constant and the number of drug molecules that are involved in binding of one β-CN molecule were calculated from plots of the fluorescence intensity at 332 nm, expressed as the percentage of the fluorescence of the drug-free β-CN vs. the added drug concentration. The data was analyzed using Matlab (MathWorks), by means of the following equations:

$$\begin{matrix}F = \frac{{F}_{0} [ {P}_{F} ] + {F}_{1} [ PL ]}{[ {P}_{F} ] + [ PL ]} & ( 1 ) \\ {K}_{d} = \frac{1}{{K}_{a}} = \frac{[ {P}_{F} ] [ {L}_{F} ]}{[ PL ]} & ( 2 )\end{matrix}$$

where F is the fluorescence intensity at a given added ligand (drug) concentration; F₀ the fluorescence intensity at the beginning of the titration; F₁ the fluorescence at the end of the titration; [P_{F}] the concentration of the free β-CN; [L_{F}] the concentration of the free ligand, drug; [PL] the concentration of the β-CN-drug complex; K_{d} and Kₐ are the dissociation and association constants respectively (Christiaenset al. 2002 European Journal of Biochemistry 269 [12]:2918-2926).

Emission spectra of pure 1 mg/ml β-CN vs. those of drug encapsulated in 1mg/ml at 4:1 drug: β-CN molar ratio, were collected at Trp excitation wavelength 287 nm, using the Fluorolog 3-22 spectrofluorometer.

Mitoxantrone Fluorescence:

The β-CN-mitoxantrone interaction was further studied by monitoring the changes in the mitoxantrone fluorescence emission upon addition of β-CN. For this purpose a 3-D fluorescence spectra analysis of mitoxantrone was performed using the Fluorolog 3-22. Mitoxantrone fluorescence emission was measured using 609 nm and 675 nm excitation and emission, respectively, and slit widths of 1 nm. Intrinsic fluorescence of 42 μM mitoxantrone was measured before and after addition of different amounts of β-CN. The β-CN concentration range was 0.1-3.8 mg/ml. The apparent dissociation constant and the number of β-CN molecules bound per mitoxantrone molecule were calculated from plots of the fluorescence intensity at 675 nm expressed as the percentage of the fluorescence of the β-CN-free mitoxantrone vs. the added β-CN concentration. Data were analyzed as detailed herein above for Trp quenching.

Visual Appearance:

In order to evaluate the visual appearance of the tested drug when encapsulated within the β-CN system as compared to the non-encapsulated drug solubilized at a similar concentration, in PBS and DMSO (i.e. without the addition of β-CN), photographs were taken of both solutions and their appearance was compared.

### Experimental Results

### Mitoxantrone Encapsulation in β-CN Particles

Particle Size Analysis:

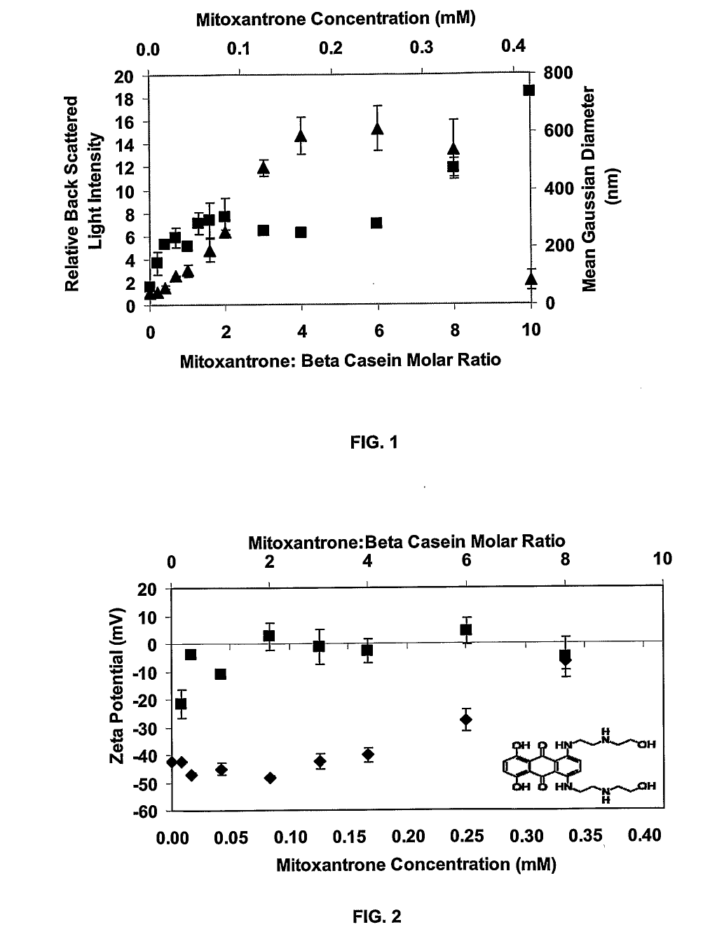

The data obtained for the Mean Gaussian diameter and back scattered light intensity as a function of mitoxantrone:β-CN molar ratio are presented in FIG. 1. As shown in FIG. 1, a constant increase in β-CN-mitoxantrone particle size was observed at mitoxantrone:β-CN molar ratio of up to 2:1. At molar ratios between 2:1 and 6:1, the number of particles increased but the diameter of the particles remained constant. At mitoxantrone: β-CN molar ratio above 6:1, the size of the particles started to increase again and a decrease in back scattered light intensity was observed.

Zeta Potential Analysis:

Zeta potential measurements of pure mitoxantrone solutions in PBS at different concentrations (8-333 μM) vs. the nano-encapsulated mitoxantrone in 1 mg/ml β-CN at same concentrations (mitoxantrone:β-CN molar ratios of from 0.2:1 to 8:1) are presented in FIG. 2. The results demonstrate that in the concentration range studied, mitoxantrone in PBS showed zeta potential values close to zero suggesting that it is colloidally unstable, and hence tends to aggregate. However, in the presence of 1 mg/ml β-CN, much more stable systems were observed. As the pI of β-CN is 5.33, it is negatively charged at pH 7.0, and the zeta potential measured was about −42 mV. As mitoxantrone:β-CN ratio was raised up to about 4:1, the zeta potential remained rather constant around −42 mV. However, as the ratio increased beyond that, the zeta potential started rising, approaching a value of zero just above 8:1 mitoxantrone:β-CN ratio. Shown in the subset of FIG. 2 is the chemical structure of mitoxantrone. The four secondary amines in mitoxantrone, are apparently responsible for the slight positive charge of this molecule at pH 7.0, (having pKa values of 5.99 and 8.13). The fact that only at a ratio of 6:1 and above, a significant rise of the zeta potential was observed suggests that mitoxantrone entrapment within the particles core is favorable compared to mitoxantrone binding to the outer particles surface.

Without being bound by any particular theory it is interpreted that mitoxantrone binds first to β-CN micelles core due to hydrophobic interactions and then, when the hydrophobic core is loaded to a maximum, mitoxantrone starts binding to the surface of the β-CN nanoparticles. The surface binding may be through electrostatic interactions to the negatively charged β-CN particle's surface (formed by the serine-phosphate groups in the hydrophilic N-terminal domain). It is further interpreted that the electrostatic interactions cause some of the β-CN micelles to aggregate with one another, to thereby form cluster-like particles in which mitoxantrone is entrapped within the hydrophobic core of the particles, as well as between the clustered micellar nanoparticles, and thus is still shielded from the external aqueous solution.

The following encapsulation mechanism of mitoxantrone within the β-CN micelles, based on the zeta potential measurements, is therefore suggested:

First, at mitoxantrone:β-CN molar ratio lower than 4:1, the zeta potential remains constant and equal to that of unloaded β-CN micelles, suggesting encapsulation of mitoxantrone within the p-CN nanoparticles. At mitoxantrone:β-CN molar ratio higher than 4:1, the nanoparticle's core is fully loaded with mitoxantrone and due to hydrophobic and electrostatic interactions, upon further addition of mitoxantrone, adherence of the drug to the outer surface of the particles is effected, thus causing an increase in the zeta potential.

Trp Fluorescence:

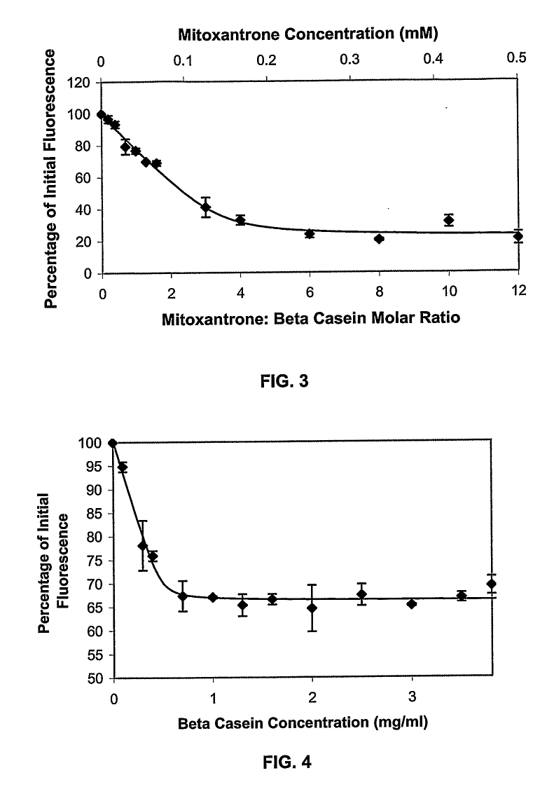

As mentioned hereinabove, Trp 143 is located in the hydrophobic domain of β-CN. Quenching of Protein fluorescence due to energy transfer from Trp to the bound ligand serves to determine the binding affinity. Trp excitation and emission wavelengths were 287 and 332, respectively. FIG. 3 shows a decrease in Trp emission intensity as a function of mitoxantrone: β-CN molar ratio. Trp emission reached a plateau at mitoxantrone:β-CN molar ratio of 6:1, at which concentration all of the accessible Trp 143 residues are apparently binding mitoxantrone. Evidently, the mitoxantrone quenches the over-all Trp emission to 20% of its initial intensity. The dissociation constant (K_{d}) and the number of mitoxantrone molecules which are involved in binding to β-CN, per protein molecule, were calculated from the model fit and the results are presented in Table 1.

Mitoxantrone Fluorescence:

Mitoxantrone is a fluorescent antitumor agent with an optimal excitation-emission wavelength pair at 609 nm and at 675 nm, respectively, according to a 3D fluorescence spectra analysis. Hence, mitoxantrone quenching by β-CN was studied. Light scattering intensity of mitoxantrone-β-CN nano particles at a constant mitoxantrone concentration of 42 μM and varying β-CN concentration was studied. FIG. 4 presents the emission intensity and of mitoxantrone at variable concentrations of β-CN as a function of β-CN concentration. FIG. 4 reveals that β-CN quenches the over-all mitoxantrone emission up to 65% of its initial intensity. Mitoxantrone emission reached a plateau at a β-CN concentration of 0.7 mg/ml, a concentration at which all of the mitoxantrone molecules are apparently entrapped within β-CN nano particles. The dissociation constant Kd and the number of β-CN molecules involved in binding of mitoxantrone were calculated from the model fit and the results are presented in Table 1.

TABLE 1

| Molecule | | | |
|---|---|---|---|
| (fluorophore) | | n (moles of | |
| whose | | quencher | Calculated |
| fluorescence is | Calculated Kd | per moles of | mitoxantrone:β- |
| quenched | (M) | fluorophore) | CN molar ratio |
| Trp | (8.43 ± 6.06) * 10⁻⁶ | 3.28 ± 0.03 | 3.28 ± 0.03 |
| mitoxantrone | (7.26 ± 2.85) * 10⁻⁷ | 0.46 ± 0.08 | 2.22 ± 0.39 |

The association of the ligand, mitoxantrone, to β-CN was found to be of high affinity, as seen from the very low dissociation constant values, which were determined independently by the fluorescence quenching of the two fluorophores, Trp 143 of β-CN, and mitoxantrone (8.43±6.06×10⁻⁶ and 7.26±2.85×10⁻⁷, respectively, see Table 1). These values are in reasonable agreement considering the independence of the two probes, and the fact they were obtained during two separate sets of experiments.

The calculated values of mitoxantrone: β-CN molar ratio (see, Table 1) were also relatively similar. These values suggest that the observed stoichiometric binding ratio between the two molecules was between 2.2-3.3 moles of mitoxantrone per mole of protein.

The actual maximal mitoxantrone drug loading of the nanoparticles was higher (6:1) than the calculated values, as obtained from the DLS particle size, zeta potential and Trp quenching analysis (see, FIGS. 1, 2 and, 3 respectively).

It is suggested that higher loading of about 6:1 mitoxantrone:β-CN may possibly be facilitated by the formation drug droplets within the particle. Beyond this ratio, the results suggest that the drug-saturated nanoparticles cannot contain anymore mitoxantrone.

In summary, the results presented hereinabove confirm that mitoxantrone is encapsulated within β-CN nano-particles with high affinity. The optimal stoichiometric mitoxantrone loading of a β-CN nano-particle system containing 1 mg/ml β-CN is between 2.2-4.0 moles of mitoxantrone per mole of β-CN according to mitoxantrone and Trp 143 emission quenching and to zeta potential analysis. The maximal mitoxantrone loading is apparently 6:1 (mitoxantrone: β-CN).

These results demonstrate that β-CN displays a very good binding and encapsulation capacity for this model anticancer drug, mitoxantrone, and thus may serve as a useful nanoscopic vehicle for the solubilization and oral delivery of hydrophobic drugs in aqueous drug preparations.

### Vinblastine Encapsulation in β-CN Particles

### Particle Size Distribution:

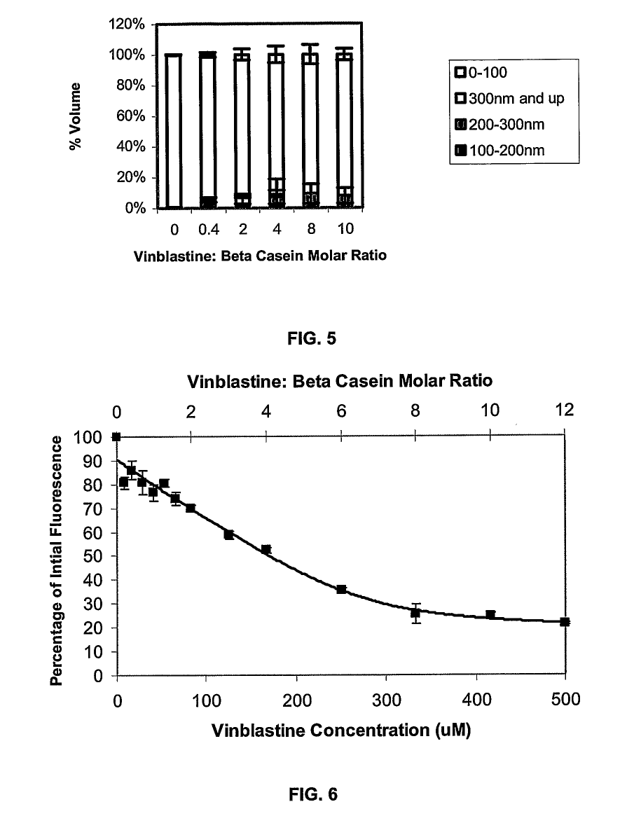

The measured particle-size distribution by volume percentage of vinblastine encapsulated within β-CN particles is shown in FIG. 5. The results show that the particle-size distribution depends on the total vinblastine:β-CN molar ratio in the solution whereby in the absence of vinblastine, the vast majority of the particles was expectedly small with an average diameter smaller than 100 nm, corresponding to pure β-CN monomers and micelles. As the vinblastine:β-CN ratio increased, the size distribution gradually shifted to larger particles due to the association and encapsulation of vinblastine within the β-CN nanoparticles.

As suggested hereinabove, the increase in particle size may be further a result of the β-CN charge neutralization, by vinblastine molecules adhered to the surface of the β-CN micelle, which results in the formation of larger aggregates.

At all the studied concentrations, more than 90% of the particles were very small, with an average diameter smaller than 100 nm.

Without being bound by any particular theory, it is suggested that the small sized β-CN particle size is due to the pKa of vinblastine being 5.5, such that at pH 7 the majority of vinblastine molecules are uncharged with only a minority of the molecules having a positive charge. Thus, because the vast of vinblastine molecules are uncharged in pH 7, the charge neutralization of negatively charged β-CN monomers by vinblastine at pH 7, is less efficient than in the case of mitoxantrone. Consequently, β-CN micellar aggregation occurs to a much smaller extent and the majority of vinblastine-loaded β-CN nanoparticles obtained are small, with an average diameter being in the range of 30-60 nm. These results point to the potential beneficial use of these small vinblastin-β-CN nanoparticles for endocytosis-mediated delivery.

Trp Fluorescence:

FIG. 6 presents the data obtained in these studies, which show a decrease in Trp-143 emission intensity at 287 nm excitation and 332 nm emission wavelengths as a function of the vinblastine:β-CN molar ratio (in the range of from 0.2:1 to 10:1 at 1 mg/ml β-CN). About 80% of the initial overall Trp emission intensity was quenched by vinblastine. The dissociation constant (K_{d}) and the number of vinblastine molecules which were involved in this association process within β-CN nanoparticles, per protein molecule, were calculated from the model fit, and were found to be (50.91±7.89)×10⁻⁶ M and 5.25±0.57 respectively.

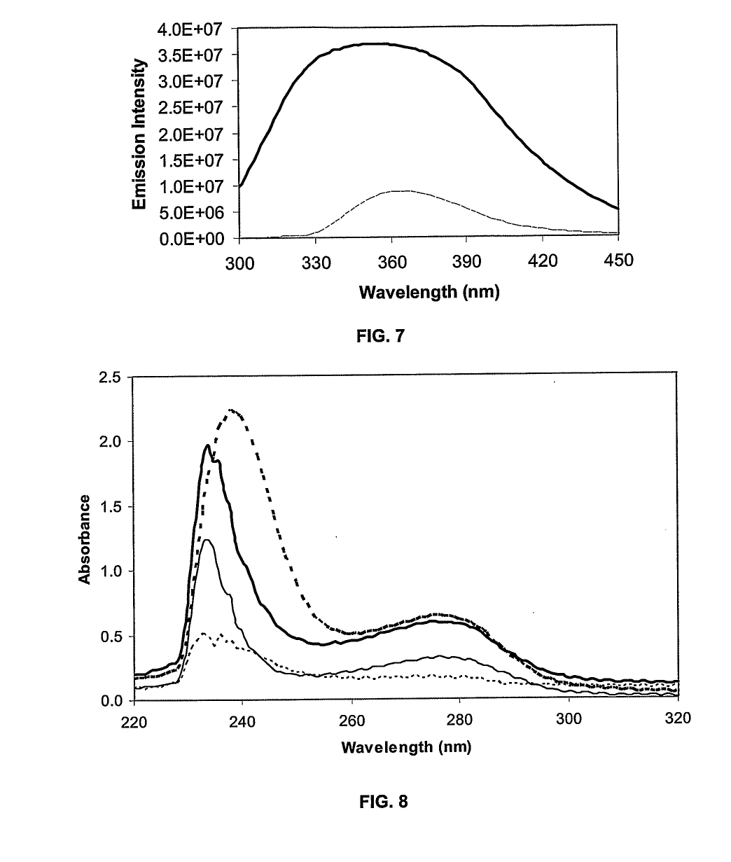

The emission spectra of pure 1 mg/ml β-CN vs. that of vinblastine-loaded 1 mg/ml β-CN at 4:1 vinblastine: β-CN molar ratio, which were collected at a Trp excitation wavelength of 287 nm is presented in FIG. 7. A red shift and a decrease in emission intensity in all measured wavelengths of β-CN's Trp-143, of vinblastine loaded β-CN nano-particles compared with pure 1 mg/ml β-CN can be observed. This decrease and red shift in Trp-143 fluorescence is due to quenching of Trp emission by vinblastine. The emission spectra data around the absolute maximum were fitted by 5^{th} degree polynomial and the wavelengths of maximum emission were determined. Compared to the peak at 354.68±0.93 nm in the pure 1 mg/ml β-CN, the peak of the spectrum of the vinblastine-encapsulated 1 mg/ml β-CN system is shifted to 364.14±0.12 nm. This shift is significant, as may be judged from the small standard error compared to the difference between these two peaks (0.93 nm vs. 9.46 nm respectively).

### Docetaxel Encapsulation in β-CN Particles

Interaction of Docetaxel with β-CN as Revealed by Absorbance Spectra Analysis:

The absorbance spectra of 168 μM pure Docetaxel, of 42 μM (1 mg/ml) pure β-CN, and of 168 μM Docetaxel encapsulated within 42 μM β-CN nanoparticles, as well as the mathematical summation of the former two spectra are shown in FIG. 8. The spectrum of absorbance of nano-encapsulated Docetaxel in β-CN at a 4:1 molar ratio differs from the sum of the pure β-CN and Docetaxel absorbance spectra. The absorbance spectra data around the absolute maximum was fitted by 5^{th} degree polynomial and the wavelength of maximum absorbance was determined. Compared to the peak at 234.24±0.21 nm in the mathematical summation plot, the peak of the spectrum of the combined system is shifted to 239.33±0.73nm. This shift is significant, as may be judged from the small standard error compared to the difference between these two peaks (0.73 nm vs. 5.09 nm respectively). This indicates that β-CN interacts with Docetaxel and that β-CN-Docetaxel combined assemblies are formed.

Zeta Potential Analysis:

Zeta potential measurements of pure Docetaxel solutions in PBS at different concentrations (83-416 μM) vs. encapsulated Docetaxel in 1 mg/ml β-CN at the same concentrations (Docetaxel:β-CN molar ratios in the range of from 0.4:1 to 10:1) are presented in FIG. 9. As shown in FIG. 9, the concentration range studied, Docetaxel in PBS showed zeta potential values around −30 mV, suggesting that it is colloidally unstable, and hence tends to aggregate (measured using Standard Test Methods for Zeta Potential of Colloids in Water and Waste Water, American Society for Testing and Materials (ASTM) Standard D 4187-82. 1985.). However, in the presence of 1 mg/ml β-CN, much more stable systems were observed, having zeta potential values of between −50 mV and −65 mV.

Visual Appearance:

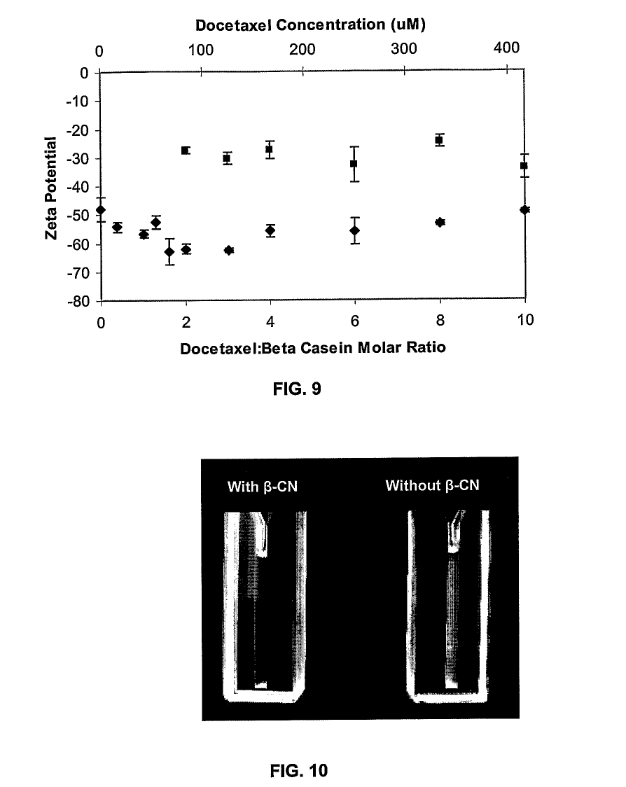

FIG. 10 presents a photograph of 504 μM Docetaxel encapsulated in 1 mg/ml β-CN at 12:1 molar ratio (left) and the same system, 504 μM Docetaxel in PBS and 4.55% DMSO, but without β-CN (right). As shown in FIG. 10, even at this high Docetaxel:β-CN molar ratio, a clear solution of Docetaxel encapsulated in β-CN nanoparticles was formed compared to turbid, unstable and aggregated solution of Docetaxel in PBS without β-CN. These results are in agreement with those of the zeta potential measurements discussed hereinabove, and are further evidence that β-CN stabilizes Docetaxel in aqueous solution.

### Paclitaxel Encapsulation in β-CN Nano Particles

Trp Fluorescence:

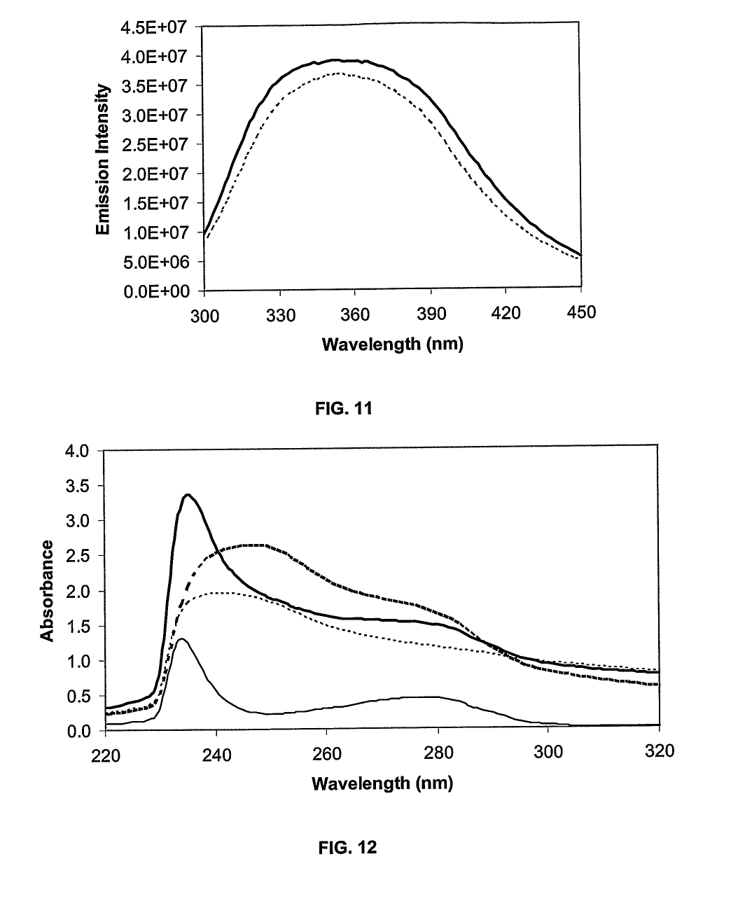

The emission spectra of β-CN Trp -143 when 1 mg/ml of β-CN is solubilized alone, as compared with the emission spectra of Paclitaxel-loaded 1 mg/ml β-CN at 4:1 Paclitaxel:β-CN molar ratio, was evaluated (287 nm) and the obtained data is shown in FIG. 11. As shown in FIG. 11, a decrease in emission intensity of β-CN's Trp-143 is observed in all measured wavelengths, upon Paclitaxel encapsulation within the β-CN nano-particles, due to quenching of the Trp-143 emission by Paclitaxel.

Interaction of Paclitaxel with β-CN as Revealed by Absorbance Spectra Analysis:

FIG. 12 presents the absorbance spectra of 168 μM pure Paclitaxel, of 42 μM (1 mg/ml) pure β-CN, and of 168 μM paclitaxel encapsulated in 42 μM β-CN, as well as the mathematical summation of the former two spectra. As shown in FIG. 12, the absorbance spectrum of nano-encapsulated paclitaxel in β-CN at a 4:1 molar ratio differs from the sum of the pure β-CN and paclitaxel absorbance spectra. The absorbance spectra data around the absolute maximum was fitted by 5^{th} degree polynomial and the wavelength of maximum absorbance was determined. Compared to the peak at 235.27±0.22 nm in the mathematical summation plot, the peak of the spectrum of the combined system is shifted to 246.67±0.67nm. This shift is significant, as may be judged from the small standard error compared to the difference between these two peaks (0.67 nm vs. 11.39 nm, respectively). This indicates that β-CN interacts with paclitaxel and that β-CN-paclitaxel combined assemblies are formed.

Visual Appearance:

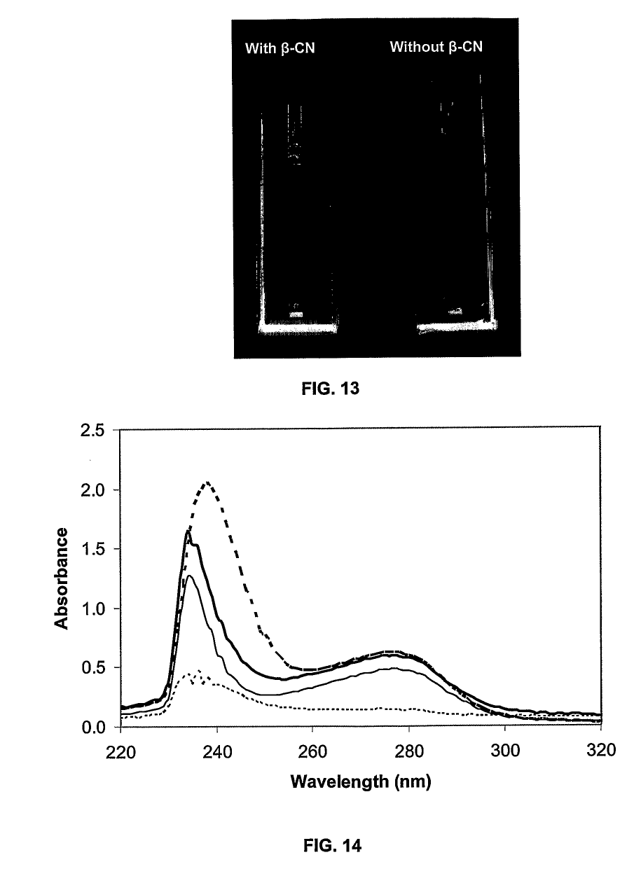

FIG. 13 presents a photograph of 84 μM Paclitaxel encapsulated in 1 mg/ml β-CN at 2:1 molar ratio (left) and a photograph of the same system of 84 μM Paclitaxel in PBS and 0.8% DMSO but without β-CN (right). As shown in FIG. 13, at this Paclitaxel:β-CN molar ratio a clear solution of paclitaxel encapsulated in β-CN nanoparticles is formed, whereby a turbid, unstable and aggregated system is formed with Paclitaxel in PBS without β-CN. This is further evidence that β-CN binds and stabilize Paclitaxel in aqueous solution.

### Irinoteacan Encapsulation in β-CN Nano Particles

Interaction of Irinotecan with β-CN as Revealed by Absorbance Spectra Analysis

The absorbance spectra of 168 μM pure irinotecan, of 42 μM (1 mg/ml) pure β-CN, and of 168 μM irinotecan encapsulated in 42 μM β-CN, as well as the mathematical summation of the former two spectra are presented in FIG. 14. As shown in FIG. 14, the absorbance spectrum of nano-encapsulated irinotecan in β-CN at a 4:1 molar ratio differs from the sum of the pure β-CN and irinotecan absorbance spectra. The absorbance spectra data around the absolute maximum was fitted by 5^{th} degree polynomial and the wavelength of maximum absorbance was determined. Compared to the peak at 234.91±0.06 nm in the mathematical summation plot, the peak of the spectrum of the combined system is shifted to 237.81±0.11 nm. This shift is significant, as may be judged from the small standard error compared to the difference between these two peaks (0.11 nm vs. 2.9 nm, respectively). This indicates that β-CN interacts with irinotecan and that β-CN-irinotecan combined assemblies are formed.

Zeta Potential Analysis:

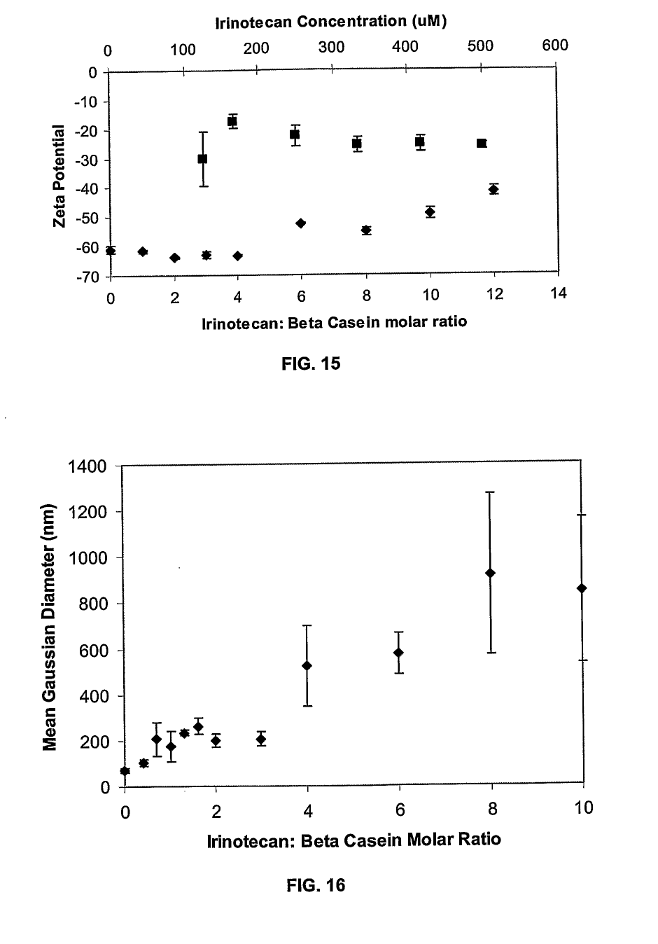

Zeta potential measurements of pure irinotecan solutions in PBS at different concentrations (125-500 μM) vs. encapsulated irinotecan in 1 mg/ml β-CN at the same concentrations (irinotecan:β-CN molar ratio in the ranges of 1:1 to 12:1) are presented in FIG. 15. As shown in FIG. 15, in the concentration range studied, irinotecan in PBS showed zeta potential values less negative than −40 mV (i.e. between −30, and −15 mV), suggesting that it is colloidally unstable, and hence tends to aggregate (measured using Standard Test Methods for Zeta Potential of Colloids in Water and Waste Water, American Society for Testing and Materials (ASTM) Standard D 4187-82. 1985.). However, in the presence of 1 mg/ml β-CN much more stable systems were observed.

As discussed hereinabove, β-CN is negatively charged at pH 7.0, and the zeta potential measured was about −60 mV. As irinotecan: β-CN ratio was raised up to about 4:1, the zeta potential remained rather constant around −60 mV. However, as the ratio increased beyond that, the zeta potential started rising. The pKa of irinotecan is 8.1 hence at pH 7.0 it has a slight positive charge. As discussed hereinabove, the fact that only at a ratio of 6:1 and above, a significant rise of the zeta potential was observed suggests that irinotecan encapsulation within the particles core is favorable compared to irinotecan binding to the outer particles surface. As in the case of mitoxantrone, it is further interpreted that irinotecan binds first to β-CN micelles core due to hydrophobic interactions and then, when the hydrophobic core is loaded to a maximum, irinotecan starts to bind to the β-CN surface via electrostatic interactions with the negatively charged β-CN particle's surface (formed by the serine-phosphate groups in the hydrophilic N-terminal domain). These electrostatic interactions neutralize the negative charge on the β-CN micelles, and lead to aggregation of some of the β-CN particles with one another, thereby forming cluster-like particles wherein irinotecan is entrapped within the hydrophobic core of the particles, as well as between the clustered micellar particles, and thus is still shielded from the exterior aqueous solution.,

Thus, it is suggested that the zeta potential remains constant and equal to unloaded β-CN micelle up to irinotecan: β-CN molar ratio of 4:1. Above this molar ratio, the particle's core is fully loaded with irinotecan and irinotecan starts to adhere to the outer particle's surface, which causes the observed increase in zeta potential.

Particles Size Analysis:

The Mean Gaussian diameter as a function of irinotecan:β-CN total molar ratio in the solution is presented in FIG. 16. The results show that up to irinotecan: β-CN molar ratio of 4:1 the particles diameter was constant with an average diameter of about 200 nm. Above this molar ratio, larger irinotecan-β-CN particles were formed possibly due to the hereinabove discussed β-CN micellar aggregation. These results are in agreement with the zeta potential measurements of irinotecan-β-CN nano particles.

Visual Appearance:

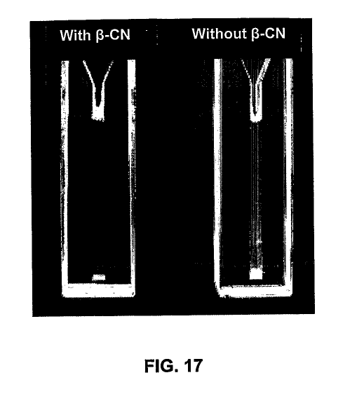

FIG. 17 presents a photograph of 504 μM irinotecan encapsulated in 1 mg/ml β-CN at 12:1 molar ratio (left) and a photograph of the same system, 504 μM irinotecan, in PBS and 5.6% DMSO but without β-CN (right). As shown in FIG. 17, even at this high irinotecan: β-CN molar ratio, a clear solution of irinotecan encapsulated in β-CN nanoparticles is formed, whereby a turbid, unstable and aggregated solution of irinotecan is formed in PBS without β-CN. These results are in agreement with those of zeta potential shown above, and provide further evidence that β-CN stabilize irinotecan in aqueous solution.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-39. (canceled)

40. A casein particle having encapsulated therein a therapeutically active agent, the therapeutically active agent being a chemotherapeutic agent.

41. The casein particle of claim 40, wherein said chemotherapeutic agent is selected from the group consisting of a hydrophobic chemotherapeutic agent having a Log P value that ranges from 1 to 10.

42. The casein particle of claim 40, wherein said chemotherapeutic agent has a water solubility lower than 1% w/v.

43. The casein particle of claim 40, wherein said chemotherapeutic agent is selected from the group consisting of paclitaxel, docetaxel, sn-38, irinotecan, doxorubicin (neutral), fluorouracil, bortezomib, camptothecin, carmustine, cisplatin, dactinomycin, docetaxel, floxuridine, ifosfamide, irinotcan, letrozole, mitomycin c, mitoxantrone, oxaliplatin, plicamycin, teniposide, valrubicin, vinblastine, vincristine and combinations thereof.

44. The casein particle of claim 40, wherein said chemotherapeutic agent is selected from the group consisting of mitoxantrone, vinblastine, docetaxel, paclitaxel and irinotecan.

45. A casein particle having encapsulated therein a therapeutically active agent that is administered parenterally if non-encapsulated and is not suitable for oral administration if not encapsulated, said therapeutically active agent being a hydrophobic therapeutically active agent and/or a water-insoluble therapeutically active agent and/or a therapeutically active agent having a water solubility lower than 1% w/v.

46. The casein particle of claim 40 wherein said casein is selected from the group consisting of beta casein, kappa casein and alpha casein.

47. The casein particle of claim 40, wherein said casein is β-casein.

48. The casein particle of claim 40 having an average diameter lower than 800 nm.

49. The casein particle of claim 40, having an average diameter lower than 100 nm.

50. The casein particle of claim 40 being is a form selected from the group consisting of a micelle, a clustered micelle, a micellar cluster, a microparticle, a nanoparticle, a cluster of microparticles, a cluster of nanoparticles, a microcluster, a nanocluster, an aggregate, a microaggregate, a nanoaggregate, a particulate, a microparticulate, and a nanoparticulate.

51. The casein particle of claim 47 wherein a molar ratio of said therapeutically active agent to β-casein monomers forming the casein particle ranges from 1:1 to 20:1.

52. The casein particle of claim 40, further comprising a targeting moiety being attached to a surface thereof.

53. The casein particle of claim 40, further comprising at least one additional agent being encapsulated therein or attached to a surface thereof.

54. The casein particle of claim 53, wherein said additional agent and said therapeutically active agent act additively or in synergy.

55. The casein particle of claim 40, being for orally delivering said therapeutically active agent.

56. A pharmaceutical composition comprising the casein particle of claim 40 and a pharmaceutically acceptable carrier.

57. A pharmaceutical composition comprising the casein particle of claim 45 and a pharmaceutically acceptable carrier.

58. The pharmaceutical composition of claim 56, comprising a plurality of said casein particles.

59. The pharmaceutical composition of claim 56, being formulated for oral administration.

60. A method of treating cancer, the method comprising administering to a subject in need thereof the pharmaceutical composition of claim 56.

61. The method of claim 60, wherein said administering is effected orally.

62. A method of treating a medical condition treatable by said therapeutically active agent, the method comprising administering to a subject in need thereof the pharmaceutical composition of claim 57.

63. The method of claim 62, wherein said administering is effected orally.
